(19) United States
(12) Patent Application Publication
(10) Pub. No.: US 2004/0142476 A1
(43) Pub. Date: Jul. 22, 2004

(54) Organellar targeting of RNA and its use in the interruption of environmental gene flow

(75) Inventors: Thomas C. Evans, Somerville, MA (US); Sriharsa Pradhan, Wenham, MA (US)

Correspondence Address: NEW ENGLAND BIOLABS, INC., 32 TOZER ROAD, BEVERLY, MA, 01915, US

(73) Assignee: New England Biolabs, Inc.

(21) Appl. No.: 10/698,630

(22) Filed: Oct. 31, 2003

Related U.S. Application Data
(60) Provisional application No. 60/423,341, filed on Nov. 1, 2002.

(51) Int. Cl. A01H 1/00; C12N 15/82

(52) U.S. Cl. 435/468; 800/282

(57) ABSTRACT

Methods and compositions are provided which relate to translocating an RNA corresponding to a gene or gene fragment into the chloroplast by means of a chloroplast localization sequence. Additionally plant cells are described in which the chloroplast contains a ribozyme fused at one end to an RNA encoding a fragment of the protein such that the ribozyme can trans-splice the translocated fusion RNA to the RNA encoding the gene fragment to form an intact mRNA encoding a functional protein.

### CROSS REFERENCE

This application claims priority from Provisional Application Serial No. 60/423,341 herein incorporated by reference

### BACKGROUND

There has been considerable concern and debate in Europe and the United States about the environmental consequences of genetically modified plants and the transmission of genetic modifications from the target plants to non-target plants by cross-pollination. This process has been characterized as gene flow.

Genetically modified plants, such as soybean, corn, cotton, or canola which are herbicide-resistant have been developed and utilized throughout the Americas and Asia. Nonetheless, concern has been raised in various countries about the unwanted spread of the genetic modifications into neighboring unmodified plants. In certain agriculturally important plants, such as corn, the pollen contains only nuclear genomic material.

A method of interrupting gene flow from transgenic plants to non-transgenic organisms would be ecologically safer and environmentally advantageous.

### SUMMARY

Interrupting gene flow in the environment can be achieved by preventing the transmission of transgenic material in pollen. Pollen from various agricultural plants contain nuclear DNA but little or no chloroplast DNA. Transmission of a particular gene between plants can be prevented if only part or none of the gene is located in nuclear DNA of such pollen. Present embodiments of the invention describe how this can be achieved by splitting a gene into fragments such that one fragment is located in the nucleus and one fragment is located in the chloroplast of the plant cell. Alternatively, RNA may be introduced into the cytoplasm of the cell via a viral vector or other vehicle.

Embodiments of the invention rely on the ability of RNA that is transcribed from a nuclear DNA fragment or is introduced via a viral vector or other means into the cell to be translocated into the chloroplast. In the chloroplast, the translocated RNA becomes spliced to an RNA fragment generated from a DNA fragment that is already contained therein. The desired protein is then translated in the chloroplast from the spliced RNA. Translocation of RNA into the chloroplast is achieved by means of a chloroplast localization sequence (CLS) derived from or corresponding to a viroid.

A method is provided for translocating an RNA into a chloroplast, where the method includes: contacting the chloroplast with an RNA comprising a first RNA sequence and a second RNA sequence, the first RNA sequence consisting of a CLS, the second RNA sequence not naturally associated with the first RNA sequence; and allowing the RNA to be translocated into the chloroplast. The RNA may further include an untranslated region (UTR) sequence located between the first RNA sequence and the second RNA sequence.

In preferred embodiments, the CLS shares substantial homology with a viroid sequence. Alternatively, the CLS consists of at least part of a viroid sequence. The viroid may be an Avsunvirodiae viroid, for example, an Avocado Sunblotch Viroid (ASBVd), a Peach Latent Mosaic Virus (PLMVd), a Chrysanthemum Chlorotic Mottle Viroid (CChMVd) or an Eggplant Latent Viroid (ELVd).

In a preferred embodiment, the second RNA sequence in the RNA has a length of less than 10 kb. Additionally, it may encode a whole or a part of a protein. For example, the protein may be a herbicide-resistant protein more particularly selected from 5-enolpyruvylshikimate-3-phosphate synthase (EPSPS) and acetolactate synthase (ALS). The protein may be an insecticidal toxin for example, a Bacillus thurigensis toxin. Alternatively, the protein may be a marker protein, for example, a green fluorescent protein (GFP) or a metabolic enzyme such as fructose 1,6-bisphosphate aldolase.

In preferred embodiments, the RNA is a product of transcription of a DNA or a product of RNA replication. More particularly, the DNA may be located in the nucleus of a plant cell containing a chloroplast or in the cytoplasm of a plant cell containing a chloroplast. The DNA may be introduced into the plant cell by a viral vector or by a physical or chemical means.

Trans-splicing occurs between RNA transported into the chloroplast and a ribozymal RNA already present in the chloroplast. In a preferred embodiment, the ribozyme is a self-splicing group I ribozyme. In particular, the ribozyme may be a Tetrahymena thermophila intron I trans-splicing ribozyme.

A third RNA sequence encoding part of a second protein may be additionally included in the RNA for transfer into the chloroplast. The third RNA sequence may be trans-spliced by means of a second ribozyme RNA to an additional RNA in the chloroplast for translation into the second protein in the chloroplast. An example of a second protein includes a replicase which is capable of enhancing production of the second protein.

In a preferred embodiment of the invention, a method for expressing a whole or a part of a protein in a chloroplast is provided which includes contacting the chloroplast with an RNA that includes a first RNA sequence and a second RNA sequence. The first RNA sequence is a CLS, and the second RNA sequence encodes a whole or part of the target protein. The whole or part of the target protein can be expressed in the chloroplast after RNA trans-splicing.

In an embodiment of the invention, an RNA is provided which includes a first RNA sequence which is substantially homologous to a segment of an ASBVd and is characterized by chloroplast localizing activity and a second RNA sequence which, when translated, corresponds to part or all of a protein. The segment of the ASBVd corresponds to at least 100 nucleotides. Alternatively, an RNA is provided in which a first RNA sequence corresponds to a viroid and is characterized by a CLS, and a second RNA sequence which, when translated, corresponds to part or all of a protein.

Embodiments of the invention further include any of a bacterial cell, a plant cell, a virus or a plasmid containing an RNA which corresponds to a viroid or includes a sequence substantially homologous to a segment of an ASBVd, the RNA having a first chloroplast localizing sequence, and a second RNA sequence which when translated corresponds to part or all of a protein. In particular embodiments, the protein is selected from a herbicide-resistant protein, a pesticide-resistant protein, a marker protein and a metabolic enzyme.

In a preferred embodiment of the invention, a method of expressing a protein in a plant is provided so that undesired gene flow in the environment is prevented. The method includes (a) introducing into the nucleus of the plant, a first DNA wherein the first DNA comprises a first DNA sequence and a second DNA sequence such that the first DNA sequence is transcribed to form a first RNA sequence having a CLS and the second DNA sequence is transcribed to form a second RNA sequence encoding a first part of a protein; (b) introducing into the chloroplast of the first plant, a second DNA, wherein the second DNA comprises a third DNA sequence and a fourth DNA sequence such that the third DNA sequence is transcribed to form a ribozyme and the fourth DNA sequence is transcribed to form a fourth RNA sequence encoding a second part of the protein; (c) permitting transcription of the first DNA and its translocation into the chloroplast for trans-splicing of the second RNA sequence to the fourth RNA sequence for translation into the protein; and (d) inhibiting undesired gene flow in the environment.

### BRIEF DESCRIPTION OF DRAWINGS

FIG. 1 illustrates the arrangement of gene components by splitting the exogenous nucleic acid into at least two fragments which are located in two cellular compartments, the nucleus and the chloroplast. Depicted here is the protein: EPSPS which is encoded by 2 fragments of DNA, the nuclear fragment encoding the N-terminal region of the EPSPS (EPSPS_{N}) and the chloroplast fragment encoding the C-terminal region of the EPSPS (EPSPS_{C}). The DNA encoding a CLS is fused to the gene fragment for EPSPS_{N}. The CLS-EPSPS_{N} gene fusion is integrated into the nuclear genome. The EPSPS_{C} gene fragment is fused to a gene encoding a trans-splicing ribozyme (Rib) RNA and placed into the chloroplast genome. Following transcription of the CLS-EPSPS_{N} gene fusion, the RNA is translocated to the chloroplast via the CLS where ribozyme mediated RNA trans-splicing fuses the EPSPS_{N} and EPSPS_{C} nucleotide sequences to generate the native, full length messenger RNA (mRNA). Translation of the spliced mRNA results in the production of the full length, active target protein.

FIG. 2 shows RNA trans-splicing in bacteria. This system can test whether transcription products of multiple fragments of a gene can be spliced together to form a single mRNA capable of being translated into a target protein. No cell compartment-specific factors are required in this model system. A bacterial cell is shown that contains a plasmid encoding the N-terminal region of the GFP gene (GFPn), and a second plasmid that encodes the C-terminal region of GFP (GFPc) and trans-splicing ribozyme RNA. Transcription of the DNA followed by RNA trans-splicing gives rise to RNA encoding the intact GFP. Expression of this RNA results in active detectable GFP protein. Active, full length GFP is detected by fluorescence measurements and by Western Blot analysis using anti-GFP antibody. In the absence of splicing, the separate GFP fragments are unable to produce polypeptide capable of fluorescent activity.

FIG. 3 provides a schematic representation of a Tetrahymena thermophila trans-splicing ribozyme RNA sequence fused to a 5′ end of the soluble modified GFP (smGFP)RNA (Genbank Accession No. U70495) truncated at nucleotide 477, which is a T in the DNA sequence (SEQ ID NO:1). There is an additional transcribed nucleotide sequence (depicted as N) that depends on the DNA fused to the truncated gene. The second transcript consists of the 5′ end of smGFP. The anti-sense region of the ribozyme is complementary to a region of the second transcript (SEQ ID NO:2) as shown. After trans-splicing, a full length mRNA for smGFP is generated. Translation from the ligated mRNA results in an active smGFP protein.

FIG. 4 provides a schematic representation of how nucleic acid localization in the chloroplast may be achieved by means of a viroid sequence. Intact GFP DNA is fused to a DNA encoding a CLS, for example, ASBVd and the plasmid containing the fusion product is introduced into the nucleus of a plant cell where it may integrate into the cell genome. Following transcription of the fusion product, the RNA is translocated into the chloroplast of a plant cell where translation to form active GFP occurs. The presence of GFP in the chloroplast can be determined by isolating the organelle and measuring GFP fluorescence or by confocal microscopy.

FIG. 5 shows how the GFP gene may be divided into GFP_{N} and GFP_{C} where (a) neither fragment encodes for a polypeptide capable of fluorescent activity and (b) there is a T suitable for ribozyme-related RNA trans-splicing of the 5′ gene fragment. A nucleotide sequence substantially homologous to a viroid sequence is fused to the GFP_{N} gene and serves as a CLS. The CLS-GFP_{N} gene fusion is integrated into the nuclear genome. The GFP_{C} gene fragment is fused to a trans-splicing ribozyme and placed into the chloroplast genome. Following transcription of the CLS-GFP_{N} gene fusion, the RNA transcript is translocated to the chloroplast via the CLS where ribozyme-mediated trans-splicing fuses the GFP_{N} and GFP_{C} nucleotide sequences to generate the native, full length mRNA. Translation of the spliced mRNA results in the production of active GFP (full-length GFP).

FIG. 6 shows the DNA sequence (SEQ ID NO:3) corresponding to the pathogenic single stranded RNA (+strand ) of the ASBVd (ASBVd(+)) obtained from GenBank (Accession No. J02020).

FIG. 7 illustrates schematically engineered gene fusion products of GFP and ASBVd(+) sequences for integration into the nuclear genome of N. tabacum. The gene fusion consists of four different gene sequences: Left and Right Border sequences (LB and RB) from Agrobacterium tumefaciens plasmid pPZP100 (Maliga, P., et al., Methods in Plant Molecular Biology: A Laboratory Course Manual, pub. by Cold Spring Harbor Laboratories, Inc., Cold Spring Harbor, N.Y.(1995)) required for integration of the gene fusion into the host genome by homologous recombination; DNA equivalents of the complete or partial ASBVd(+) sequence; 5′ UTR from the N. tabacum plastid encoding the rubisco large subunit; and smGFP. The fusion construct is suitable for use in the experimental system described in FIG. 5.

In experiments to determine the effectiveness of the CLS, we use as a negative control a fusion nucleic acid sequence in which the ASBVd(+) sequence has been replaced with the nucleic acid sequence encoding the chitin binding domain from Bacillus circulans (Watanabe, T., et al., J. Bacteriol. 176(15):4465-72 (1994) and Chong, S., et al., Gene 192(2):271-81 (1997)).

FIGS. 8A-8C provides the DNA sequences for 3 different ASBVd(+)-UTR-smGFP fusion constructs in which different sequences from ASBVd are used.

FIG. 8A shows a segment of the pVUG1 sequence (SEQ ID NO:4) which contains the first 164 nucleotides of ASBVd(+) corresponding to nucleotides 1-164 in the gene fusion sequence, the UTR corresponding to nucleotides 171-352 in the gene fusion sequence, and smGFP corresponding to nucleotides 353-1069 in the gene fusion sequence.

FIG. 8B shows a segment of the pVUG2 sequence (SEQ ID NO:5) which contains the last 164 nucleotides of ASBVd(+) corresponding to nucleotides 1-164 in the gene fusion sequence, the UTR corresponding to nucleotides 171-352 in the gene fusion sequence, and smGFP corresponding to nucleotides 353-1069 in the gene fusion sequence.

FIG. 8C shows a segment of the pVUG3 sequence (SEQ ID NO:6) which contains the last 82 nucleotides of ASBVd(+) fused to the first 82 nucleotides of ASBVd(+). This fusion is located at nucleotides 1-164 in the gene fusion sequence, the UTR corresponding to nucleotides 171-352 in the gene fusion sequence, and smGFP corresponding to nucleotides 353-1069 in the gene fusion sequence.

FIG. 9 shows a Western Blot illustrating the results of transfecting plant cells with fusion genes such as described in FIGS. 8A-8C and obtaining expression of a target protein. The expression of smGFP was determined following transfection of plant cells with the ASBVd(+)-UTR-smGFP fusion gene. Homogenized plant cells were subjected to Western Blot analysis using anti-GFP antibody.

Lane 1 contains the biotinylated molecular weight markers. The expected mass of the marker in kDa is shown at the left of the image.

Lane 2 is tissue from an untransfected plant as a negative control.

Lanes 3-7 correspond to samples from independent plants transfected with the fusion gene from pVUG1 showing successful transfection and expression of smGFP in lanes 3, and 5-7.

Lanes 8-9 corresponds to samples from plants transfected with the fusion gene from pVUG2 where expression of a transfected plant was achieved as shown in Lane 9.

FIG. 10 is similar to FIG. 1 except smGFP replaces EPSPS and the ribozyme is shown during splicing with the nuclear fragment of the smGFP being spliced to the chloroplast fragment of the smGFP to form an intact message with additional UTR and viroid sequences upstream from the protein translation start site. The ribozyme RNA is released.

FIG. 11 shows different uses, in the split gene methodology, of a CLS in addition to that described in FIG. 1

A) A 5′ UTR is fused to the DNA encoding an inactive N-terminal portion of a target protein (Target_{N}). The UTR-Target_{N} is located in the nuclear genome of a plant cell. A trans-splicing ribozyme fused to a gene encoding the C-terminus of a target protein (Target_{C}) is located in the chloroplast genome. A CLS present in the cytoplasm interacts with the UTR-Target_{N} transcript via complementary base pairing. The CLS can be delivered to the cytoplasm by encoding its DNA equivalent in the plant genome, by a virus or viroid, by transfection of the RNA or its DNA equivalent, by biolistic approaches, or by A. tumefaciens. The CLS chaperones the nucleotide sequence to the chloroplast where an active target protein is generated as described in FIG. 1.

B) The CLS-UTR-Target_{N} sequence is introduced into the plant cell by a virus or viroid, by transfection of the RNA or its DNA equivalent, by biolistic approaches, or by A. tumefaciens. The introduced CLS-UTR-Target_{N} then is translocated to the chloroplast via the CLS where active target protein is generated as described in FIG. 1.

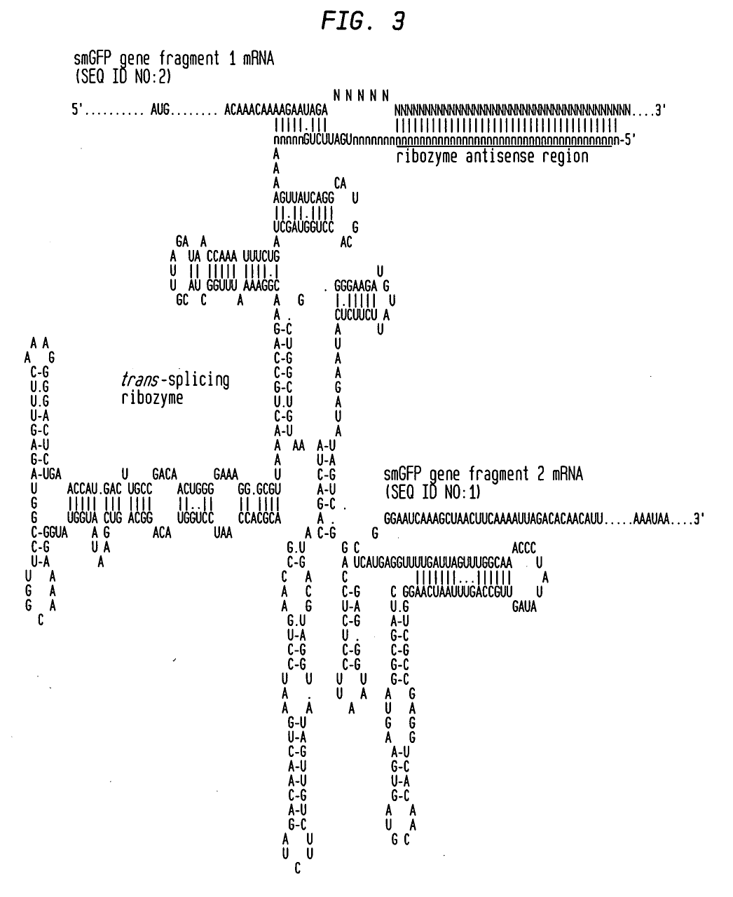

C) A CLS can potentially transport two or more sequences. In this schematic, two partial DNA sequences corresponding to target protein fragments are fused to one CLS and integrated into the nuclear genome. The CLS guides the UTR-Target1_{N}-CLS-UTR-Target2_{N} transcript into the chloroplast where ribozyme-mediated trans-splicing results in full-length transcripts which are translated to full-length Target1 and Target2 proteins. The Rib1 and Rib2 ribozymes are specific for the desired Target1_{N} or Target2_{N} RNA by designing the proper annealing sites in the 5′ end of the ribozyme as described herein (ribozyme antisence region, FIG. 3) and in (Kohler, U., et al., J. Mol. Biol., 285(5):1935-1950 (1999));

D) As described in (C) the UTR-Target1_{N}-CLS-UTR-Target2_{N} transcript can be targeted to the cholorplast. This interacts and reacts with a Rib1-Target1_{C}-Rib2-Target2_{C} transcript to generate the full-length mRNA transcripts. These are translated to full-length Target1 and Target2 proteins. The Rib1 and Rib2 are specific for the desired targets as described in (C).

E) Two or more CLS-UTR-Target DNA fusions are integrated into the nuclear genome. The CLS sequences chaperone the transcripts to the chloroplast. In the chloroplast, the corresponding ribozyme DNA fusions specifically interact and react with the translocated RNA sequences. The full-length mRNAs, formed by ribozyme-mediated trans-splicing, are translated to form the desired protein products.

F) Similar to (E) except that the two or more ribozyme DNA sequences are covalently attached and integrated into the chloroplast genome.

### DESCRIPTION OF THE EMBODIMENTS

It is desirable to prevent gene flow in the environment from organisms that have been genetically modified to those that have not. Organisms include any unicellular or multicellular eukaryotic cell or organism including plants. Throughout the description, while plants may be described and in particular, nuclear and chloroplast compartments in plant cells utilized, the methods of the invention are not limited to these compartments. For example, in addition to the nuclear or cytoplasmic compartment, another compartment of the type that contains DNA and is predominantly or completely maternally transmitted may be utilized. For example, a second compartment might be the mitochondria.

By placing gene fragments in the chloroplasts of a plant cell, those gene fragments will not be present in those pollen cells which contain predominantly nuclear genomic material. The resulting inability of a pollen-fertilized organism to synthesize the protein should significantly reduce any undesirable effects that might otherwise arise from gene flow between transgenic and non-transgenic plants

In a preferred embodiment of the present invention, a gene encoding a target protein or peptide is split into DNA fragments which are introduced into different compartments in the plant cell. Splitting a target gene into fragments located at separate loci greatly reduces the chance of inadvertent transfer of the entire protein coding sequence into other organisms. The gene product is made when the transcription products from DNA fragments located in separate compartments in a cell are brought together in a single compartment and spliced to form an intact messenger RNA which is then translated into a target protein. This approach differs from that described in U.S. patent application Ser. No. 10/377,134, which relies on protein splicing of protein fragments synthesized in different cellular compartments to assemble the target protein.

Embodiments of the method can be applied to monocistronic and polycistronic exogenous genes such as the Bacillus thurigensis toxic protein operon. For polycistronic genes, it may be advantageous to form a plurality of gene fragments for distribution between different compartments in the cells of an organism. Additional embodiments include forming transgenic plants expressing more than one type of exogenous protein. For example, gene fragments which cumulatively encode a pharmaceutical protein and replicase may be cotransformed into plant cells (U.S. Pat. No. 5,824,856).

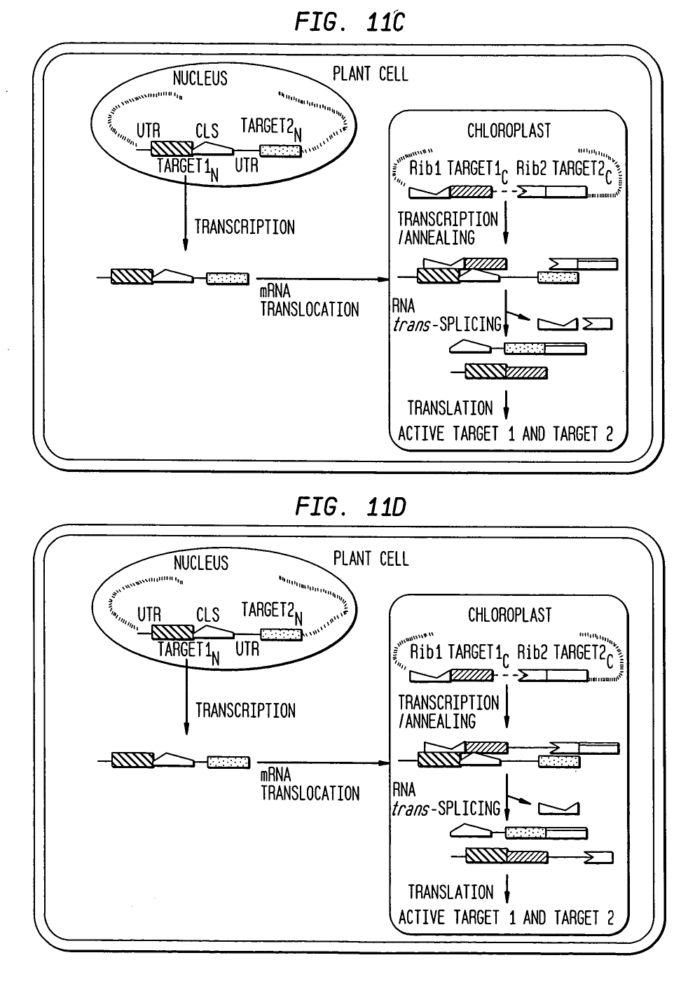

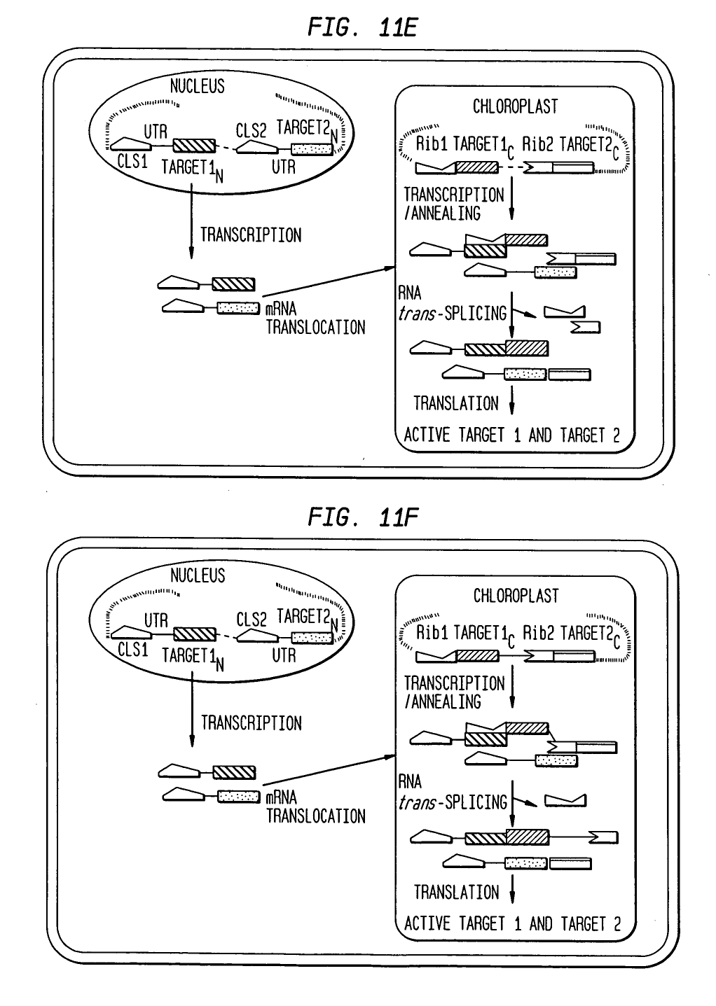

Reconstitution of a protein by splicing transcripts encoding protein fragments (see for example, FIGS. 1, 2, 5 and 11) offers advantages over reconstitution of a protein from protein fragments. For example, protein trans-splicing relies on determining an appropriate splice site to generate the protein fragments so that the spliced protein results in an active form. This can be a time-consuming step.

In contrast, RNA trans-splicing is not dependent on the architecture of the encoded protein. Protein folding issues are avoided because gene fragments are reconstituted at the mRNA level instead of at the protein level. Following RNA trans-splicing the entire protein is translated just as if the two gene fragments had never been split (FIG. 1).

In a preferred embodiment, one or more fragments of a gene encoding the target protein are introduced into the nucleus of a plant cell together with a DNA sequence encoding a chaperone molecule for transporting the RNA transcript of the nuclear DNA fragment or fragments into the chloroplast. There are numerous art-recognized methods for introducing foreign DNA into the nucleus of plant cells. These methods include physical, chemical or biological methods of delivery. Biological methods of delivery include use of viruses or bacteria.

The chaperone molecule is preferably encoded by DNA which is fused to the gene fragment or fragments although it is envisaged that in certain situations, a DNA sequence encoding the chaperone molecule may be located at a separate site in the nucleus. For example, a gene fragment may be present on either a chromosome or on an extrachromosomal DNA. Similarly, the chaperone DNA sequence may be located on either the nuclear chromosome or on extrachromosomal DNA in a location that is independent from the gene fragment or adjacent to the gene fragment.

Preferably the chaperone molecule is an RNA molecule containing a CLS where the RNA is substantially homologous in sequence with part or whole of a viroid RNA. In those situations where the RNA chaperone has natural cleaving or splicing activity, the chaperone is modified in such a way as to inhibit the cleavage or splicing activity. For example, where the RNA chaperone has hammerhead ribozyme activity, this can be inactivated or minimized by linearizing the circular viroid RNA at a site corresponding to the ribozyme cleavage site so as to disrupt cleavage activity.

For example, if the RNA chaperone is ASBVd which has ribozyme activity, then the viroid would be linearized so that the DNA equivalent of the viroid sequence would have a 5′ GTC and a 3′CAG. The linear DNA equivalent of a viroid sequence could be created as described in Example II. For example, overlapping DNA fragments can be synthesized in vitro and then ligated together to form the desired viroid sequence. The viroid sequence may then be integrated into a plasmid vector at one or more suitable restriction endonuclease cleavage sites. The reported cleavage site of the hammerhead ribozyme is between the C and U of the sequence GUCUGU (Sano, T.; Singh, R. P., Avocado Sunblotch Viroid Group: 363-371, eds. Singh, R. P.; Singh, U. S.; Kohmoto, K., Pathogenesis and Host Specificity in Plant Diseases: Histopathological, Biochemical, Genetic and Molecular Bases (1995) so that the linear viroid DNA would not contain the hammerhead structure associated with ribozyme self-cleavage activity. In the unlikely event that the fused viroid RNA formed an active hammerhead ribozyme then only the GUC present at the viroid 5′ end would be cleaved off and lost. The remaining viroid sequence would still be part of the RNA fusion translocated to the chloroplast. The viroid sequence may be introduced into the plant cell in the form of a DNA sequence fused to a gene fragment. The fused DNA may be contained within a suitable vector or may be excised from the vector prior to transformation.

In an alternative embodiment, instead of fusing a DNA encoding an RNA chaperone to a gene fragment, the RNA chaperone molecule may reside in the cytoplasm of the host cell for interacting with the transcription product of the gene fragment or fragments. The RNA chaperone may alternatively be fused to an RNA transcript of the gene fragment and packaged in a viral capsid for infecting target cells and for introducing the fusion RNA into the cytoplasm for translocation into the chloroplast. Similar possibilities can be constructed for mitochondrial localization chaperones.

In addition to the above-specified compartmentalization of a first gene fragment or RNA transcript, a second gene fragment is additionally introduced into a second compartment in a host cell, for example, the chloroplast.

In a preferred embodiment, this second gene fragment is fused to a DNA encoding a splicing agent and introduced into a single site in the chloroplast or mitochondria or other organelle. Alternatively, the splicing agent is encoded by DNA at a separate site in the chloroplast or mitochondrial DNA from that of the gene fragments. Introduction of the DNA into chloroplasts can be accomplished by means of a gene gun or by other physical or chemical methods described herein or alternatively by viral delivery. Other art-recognized methods may additionally be used. In a preferred embodiment, the splicing agent is RNA. Preferably, the RNA is a ribozyme. The RNA-splicing agent may be in the native form or may be modified to enhance functionality. In addition to RNA-splicing agents, other splicing agents known in the art may be used.

In those circumstances where it is desirable to express two or more exogenous proteins in a single plant cell, the gene fragments encoding each protein may be introduced into the nucleus or cytoplasmic compartments as separate entities using the procedures described herein. However, other arrangements in which a single fusion contains a plurality of distinct gene fragments within, for example, the nucleus, could be achieved where appropriate splicing of each gene fragment transcription product in the fusion would occur in the chloroplast from RNA fusions of single gene fragments and a ribozyme.

There are widespread applications for transgenic plants in which gene flow has been regulated according to the methods described herein. For example, modifications of existing plants may lead to:

the introduction of novel pathways to modulate desirable or undesirable production of selected naturally occurring constituents;

increased metabolic efficiency for the plant;

new uses for plants such as bioremediation;

production of reagents in large amounts including pharmaceutical proteins; and

savings on energy cost to the cell for more efficient agriculture.

It is presently possible to produce plants with unique physiological and biochemical traits and characteristics of high agronomic and crop-processing importance. Traits that play an essential role in plant growth and development, crop yield potential and stability, and crop quality and composition include enhanced carbon assimilation, efficient carbon storage, and increased carbon export and partitioning. The present methods relying on RNA translocation into the chloroplast from the cytoplasm may be used to express structural proteins, marker proteins, receptor proteins, binding proteins, enzymes or toxins suitable for enhancing traits in the organism that are beneficial for agriculture.

Examples of uses of the present split-gene methodology include the following:

Metabolic enzymes which are capable of improving plant yield by increasing starch biosynthetic ability include, for example, Fructose 1,6-bisphosphate aldolase (see for example U.S. patent application Pub. No. US-2003-0126641-A1).

Herbicide-resistant genes encode proteins that confer resistance to chemical compounds which are designed to block vital metabolic pathways in plants. These include, for example, herbicide-resistant protein EPSPS. EPSPS confers resistance to the glyophosate herbicides. The enzyme is involved in the biosynthesis of protein building blocks during the synthesis of aromatic amino acids (Stalker, et al., J. Biol. Chem. 260:4724-4728 (1985); U.S. Pat. No. 5,188,642). The EPSPS gene can be cloned from Salmonella typhimurium.

FIG. 1 and Example IIIa illustrate how plants can be modified to become herbicide-resistant through expression of EPSPS.

Another example of a herbicide-resistant gene is ALS gene from Escherichia coli (LaRossa and Schloss, J. Biol. Chem., 259:8753-8757 (1984); Chaleff and Ray, Science, 223:1148-1151 (1984); Falco and Dumas, Genetics, 109:21-35 (1985)). ALS protects the plant from the adverse effects of the commonly used sulfonylurea herbicides (SU), such as sulfometuron methyl (SM) (Short and Colburn, Toxicol Ind. Health, 15:240-275 (1999)) which blocks the growth of bacteria, yeast and higher plants by inhibiting ALS (EC 4.1.3.18).

Transfection of organisms with a mutated ALS gene results in resistance to the SM herbicide (Hill, et al., Biochem. J., 335:653-661 (1998). Similar point mutations have been identified in the ALS genes isolated from naturally occurring resistant crops, corn, cocklebur and tobacco (Lee, et al., EMBO J., 7:1241-1248 (1988); Bernasconi et al., J. Biol. Chem., 270:17381-17385 (1995)). Some of these SU tolerant crops, such as corn IC I8532 IT and Pioneer 3180 IR have been commercialized.

An example of a protein toxin is BT toxin which is naturally produced by the bacterium Bacillus thuringensis (see for example, U.S. patent application, Pub. No. US-2003-0041353-A1. BT toxin has been found to be effective for killing insect pests that might feed from or cause a pathology to the plants for which protection is sought. BT toxin is particularly suited to the methods described herein. Commercially available transgenic corn has been genetically engineered with BT toxin. Maize is amenable to the prevention of gene flow as described herein because maize displays maternal inheritance of the chloroplast (Birky, C. W., Proc. Natl. Acad. Sci. USA, 95:11331(1995)) and therefore the pollen would transmit only nuclear DNA carrying only a non-functional fragment of BT to any weedy relatives. Example V shows how the present methods may be used to obtain BT transgenic maize. Other plants of economic importance that may be genetically engineered according to the present methods include modified corn plants, cotton plants and potato plants (see for example, U.S. patent application Pub. No. US-2003-0126641-A1).

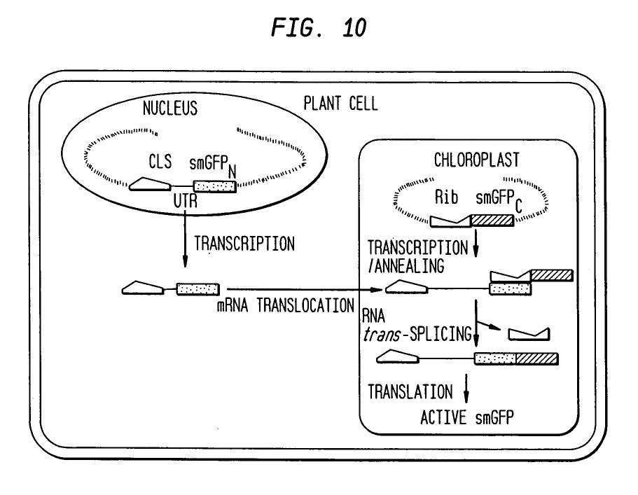

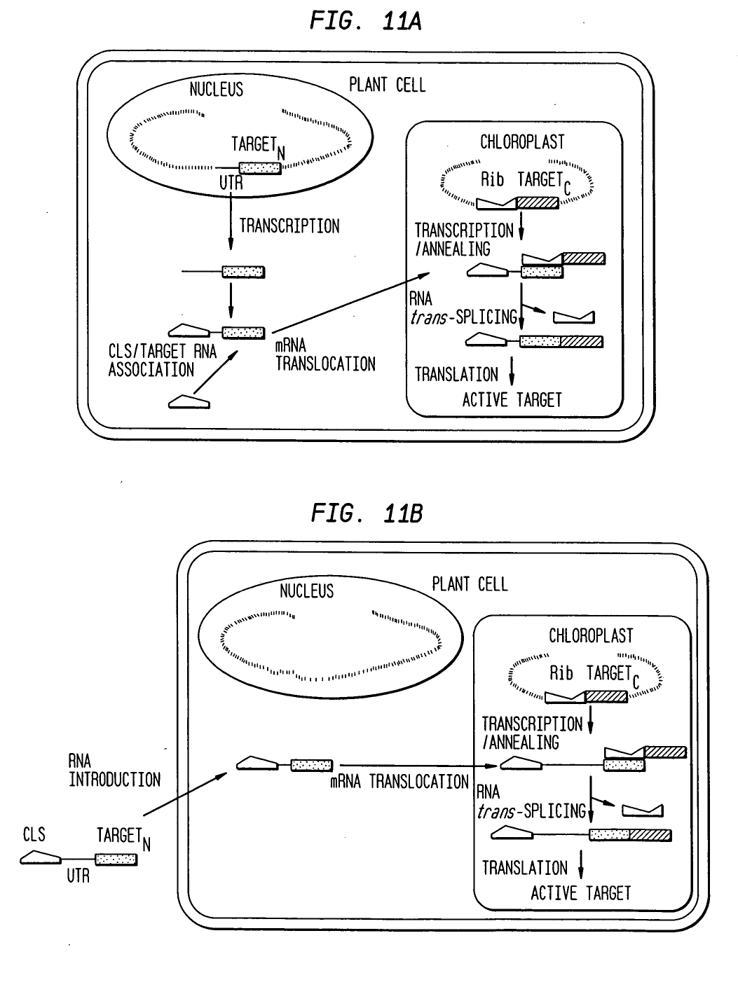

In addition to the above, modification of plants to express the marker protein, GFP, (Davis, et al., Plant Mol. Biol. 36:521-528 (1998)) is described in Example IV and FIG. 10.

In addition to regulating gene flow, an advantage of embodiments of the present invention is that the host cells expressing only one inactive fragment of a toxic target protein can be handled safely, thereby reducing the risk of exposing humans and the environment to the intact target protein, which when expressed, may be toxic, for example diptheria toxin.

The following definitions apply to the description and the claims unless the context requires otherwise.

“Trans-splicing ribozyme” refers to an RNA molecule which has catalytic activity suitable for splicing two or more RNA molecules.

Ribozyme-mediated RNA trans-splicing has been described by Long, M. B. & Sullenger, B. A., Mol. Cell. Biol., 19(10):6479-6487 (1999); Ayre, B. G., et al., Proc. Natl. Acad. Sci. USA, 96(7):3507-3512 (1999); Kohler, U., et al., J. Mol. Biol., 285(5):1935-1950 (1999); Ast, G., et al., Nuc. Acids Res., 29(8):1741-1749 (2001); Mei, R. & Herschlag, D. Biochemistry, 35(18):5796-5809 (1996); Rivier, C., EMBO J., 20(7):1765-1773 (2001).

Ribozymes for use in preferred embodiments include self-splicing group I introns. Group I introns include introns from ribosomal RNA genes such as those that are obtainable from mitochondrial genomes of fungi such as yeast, chloroplast genomes, nuclear genomes of “lower” eukaryotes, for example, ciliated protozoan, Tetrahymena thermophila or the plasmodial slime mold, Physarum polycephalum. Synthetic versions of group 1 introns which can be made in vitro can be selected in those situations where repeated splicing events are desirable. Other self-splicing introns include group II introns which are found in mRNA genes form yeast mitochondria or other fungi and in some chloroplast genomes.

In one embodiment, the ribozyme is derived from the T. thermophila group 1 intron. A uracil in one of the mRNAs to be spliced is required for the formation of a U:G mismatch to specify the splice-point residue (Kohler, et al., J. Mol. Biol. 285(5):1935-1950 (1999); Mei, et al., Biochemistry 35:5796-5809 (1996)). This ribozyme trans-splices foreign mRNAs in a number of cell types including bacterial (Kohler, et al., J. Mol. Biol. 285:1935-1950 (1999)), mammalian (Jones, et al., Nature Med. 2:643-648 (1996)) and yeast (Ayre, et al., Proc. Natl. Acad. Sci. USA 96:3507-3512 (1999)) cells. In another embodiment, the naturally occurring trans-splicing group II intron of C. reinhrdtii may be used in chloroplasts (Rivier, et al., EMBO J. 20(7):1765-1773 (2001)).

The ribozyme shown in FIG. 3 has one RNA molecule which is covalently attached at the 3′ end of the ribozyme and a second RNA molecule which has complementary sequences at the 5′ end of the ribozyme for forming an annealing association. The ribozyme catalyzes the splicing of the first RNA molecule to the second RNA molecule.

“Chloroplast localization sequence” refers to embodiments in which a first RNA sequence or molecule is capable of transporting or “chaperoning” a second RNA sequence or molecule into a chloroplast from an external environment inside a cell or outside a plastid. The CLS in an embodiment of the invention is substantially similar or complementary to an intact or partial viroid sequence. The CLS may be encoded by a DNA sequence, which is then transcribed in RNA, which in turn gives rise to the chaperone function.

The term “viroid” refers to a naturally occurring single stranded RNA molecule (Diener, Adv. Virus Res., 57:137-84 (2001); Flores, R., C. R. Acad. Sci. III. 324(10):943-52 (2001); and Flores, et al., Adv. Virus Res., 55:271-323 (2000)). Viroids are generally understood to contain between 200-500 nucleotides and in nature are generally circular molecules. Examples of viroids that contain CLS include ASBVd, PLMVd (Bussiere et al., J. Virology 6353-6360(1999)), and possibly CChMVd and ELVd. All four of these viroids are classified as Avsunviroidae and all have ribozyme activity which includes self-cleavage activity. (Flores et al., Biol. Chem. 380:849-854(1999)). Consequently, viroids in fusion genes, according to embodiments of the invention, are modified so as to inhibit the self-cleavage activity.

A preferred embodiment utilizes a modified ASBVd (Symons, Nucleic Acids Res., 9(23):6527-6537 (1981)) (Navarro, et al., Virology, 268(1):218-225 (2000); Navarro, et al., Virology, 253(1):77-85 (1999); and Lima, Arch. Virol., 38(3-4):385-390 (1994)).

A “target protein” refers to any protein of interest that is desired to be expressed in a genetically modified plant or animal.

“Substantially similar” or “substantial homology” are terms that refer to a nucleic acid sequence that hybridizes to a viroid sequence under hybridization conditions in which viroid DNA is prehybridized for 16 hours in PHS solution (6×SSC, 0.05 M sodium phosphate (pH 6.8), 1 mM EDTA, 5× Denhardt's solution, and 100 μg/mL salmon sperm DNA) at 37° C. After removing the prehybidization solution, hybridizing solution 6×SSC, 0.05 M sodium phosphate (pH 6.8), 1 mM EDTA, 5× Denhardt's solution, 100 μg/mL salmon sperm DNA, and 100 mg/mL dextran sulfate is added followed by the oligonucleotide probe to 180 pmoles. Alternatively, substantial homology refers to at least 50% homology between two nucleic acid sequences.

“Transfected” and “transformed” are used interchangeably to denote the introduction of nucleic acid into a cell or cell compartment.

The term “gene” refers to chromosomal nucleic acid, plasmid DNA, cDNA, synthetic DNA, or other DNA, RNA that encodes a peptide, polypeptide, protein, or for DNA, an RNA molecule, and regions flanking the coding sequence involved in the regulation of expression and stability of mRNA.

The term “plastid” refers to the class of plant cell organelles that includes amyloplasts, chloroplasts, chromoplasts, elaioplasts, eoplasts, etioplasts, leucoplasts, and proplastids. These organelles are self-replicating and contain what is commonly referred to as the “chloroplast genome,” a circular DNA molecule that ranges in size from about 100 kb to about 250 kb, depending upon the plant species.

Present embodiments of the invention include one or more of the following methods:

(1) identifying a suitable split site on a gene encoding the target protein;

(2) modifying a viroid RNA to remove self-cleaving activity;

(3) splitting the gene into two or more fragments and fusing each fragment to a DNA encoding a viroid RNA or a ribozyme;

(4) screening the host cell for active gene product in a bacterial assay or an in vitro assay;

(5) introducing the DNA fragments into targeted compartments within the host cells; and

(6) determining the location of split gene sequences in the relevant cellular compartment.

(1) Identifying a Suitable Split Site on a Gene Encoding the Target Protein

The split site on a gene encoding the target protein is not critical to the function of the protein expressed by the spliced messenger RNA because splicing of the fragments in the chloroplast by the ribozyme does not introduce additional nucleotides or result in the loss of nucleotides from the fragments. It is however desirable to have a U on an RNA fragment which signals ribozyme splicing.

(2) Modifying a Viroid RNA to Remove Self-Cleaving Activity

Modification of viroid RNA can be achieved by forming mutations in the viroid fragment by error prone PCR, linker scanning, site directed mutagenesis, or by mutagenic compounds and the activity of the fragments tested as described above.

Alternatively, the inhibition of self-cleaving associated with a hammerhead structure of the viroid may be achieved by linearizing the normally circular viroid near the site of viroid cleavage to prevent proper folding of the hammerhead structure.

(3) Splitting the Gene Into Two or More Fragments and Fusing Each Fragment to a DNA Encoding a Viroid RNA or a Ribozyme

Unless otherwise stated, basic DNA manipulations and genetic techniques, such as DNA cleavage, PCR, agarose electrophoresis, restriction digests, ligations, E. coli transformations, colony screens, and Western Blots are performed essentially by the protocols described in (Sambrook and Russell, Molecular Cloning: A Laboratory Manual, 2^{nd} Edition, Cold Spring Harbor Laboratory Press, Cold Spring Harbor, N.Y. (2001).

A gene encoding a target protein may be split into two or more fragments using, for example, PCR primers with appropriate restriction sites which may be designed so that one corresponds to the start of the target gene and the other to the sequence at the split site. Another set of PCR primers may be designed that correspond to the split site and the other end of the target gene. The two target gene fragments are then amplified by PCR (Sambrook, et al., supra) and cloned into a plasmid vector with the same unique cloning sites present in the PCR primers. Once cloned into separate vectors, reverse transcribed viroid or ribozyme DNA are fused to the target gene fragments.

The gene fragment fusions are then transferred to the same or separate expression vectors and transformed into bacterial or eucaryotic cells to screen for the desired activity of the target protein. The gene fragments in question could be cloned using restriction sites within or external to the viroid or ribozyme DNA present either naturally or added by mutation. Also, recombination sites may be used instead of restriction enzyme sites for the movement of the gene by recombination. The gene or gene fragments may then be transferred and/or transcribed from a plasmid vector, a viral genome or the genome of a bacterial, eucaryotic, or archeal organism in the targeted compartment of the cell.

FIG. 7 illustrates a preferred embodiment of a viroid fusion DNA in which viroid-gene fragment DNA sequences are flanked by homologous recombination sites to permit integration into the nuclear genome of a plant cell. This can be achieved using Agrobacterium tumefaciens (Zupan, Plant J. 23(1):11-28 (2000); Methods in Plant Molecular Biology: A Laboratory Course Manual” pub. Cold Spring Harbor Laboratories, Ed. by Melig et al. (1995)). Fusion constructs can further be cloned into suitable vectors using the methods described in International Application No. PCT/US03/10296.

(4) Screening the Host Cell for Active Gene Product In Bacterial Assay or In Vitro Assay

Methods for screening for expression of target proteins will vary with the target gene and the host cell. An in vitro assay may alternatively be used in which RNA fragments are spliced together in the presence of a ribozyme (Sullenger, B. A., & Cech, T. R., Nature 371:619(1994)).

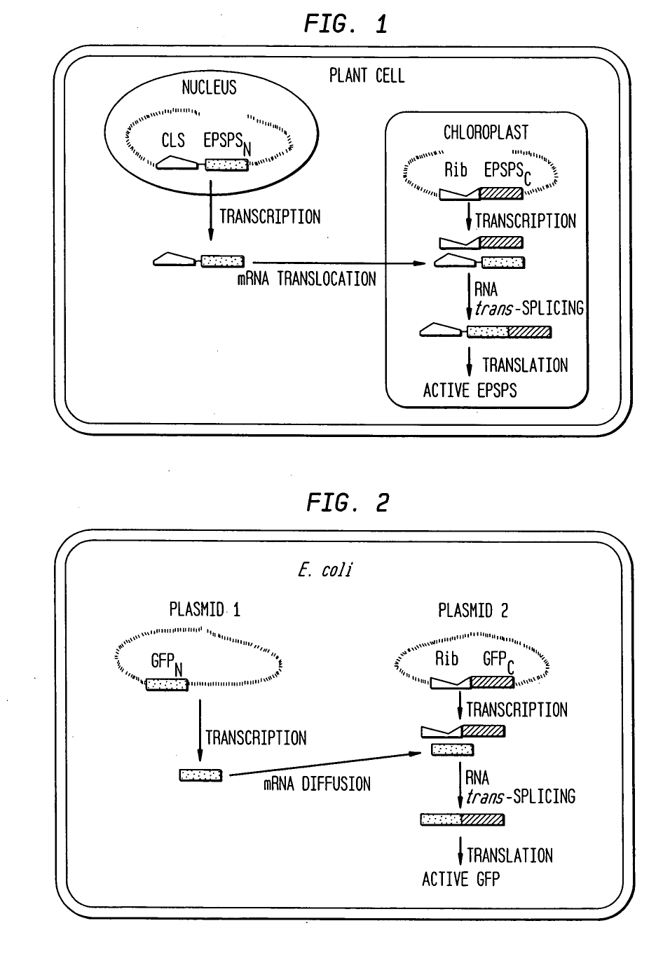

Alternatively, an in vivo assay may be used to determine protein activity biochemically or by cell phenotype, such as viability, morphology, sensitivity, or insensitivity to a drug or compound, appearance, or ability to bind or not bind a specific molecule or compound. One preferred method is to use bacteria as host cells to test, for example, herbicide-resistant activity of the re-assembled product of a split gene. FIG. 2 shows a bacterial system for assaying for protein activity after splicing without the additional complications of compartmentalization. The bacterial cells should be sensitive to the herbicide in question. The target gene fragments, with the viroid or ribozyme DNA fusion, is present on a plasmid or plasmids and is transformed into E. coli cells using standard techniques.

The gene fusions are expressed either constitutively or by an inducible promoter. E. coli are then tested for growth under selection conditions, i.e., in the presence of herbicide, in both the presence or absence of the appropriate gene fragments. Growth in the presence of the gene fragments indicates the reconstitution of the target protein activity. The E. coli cells could be substituted with any bacterial, archaea, or eucaryotic cell types (either single or multicellular) by employing techniques well known in the art.

The bacterial cell assay using GFP as the target protein as shown in FIG. 2 has been found to be well suited for rapid testing of plasmid constructs and for establishing variants on the splicing methodology which may include modifying the viroid sequence or varying the ribozyme.

(5) Introducing the DNA Fragments Into Targeted Compartments within the Host Cells

Methods of transfecting genes and gene fragments into nuclei and chloroplasts are well known in the art. For example, for genetic engineering of plant cells or plastids, see U.S. Pat. No. 5,545,818, and for genetic engineering of chloroplasts, see for example, U.S. Pat. No. 5,932,479, U.S. Pat. No. 5,693,507, International Patent Application Pub. No. WO 99/10513, U.S. Pat. No. 6,423,885 and U.S. patent application Pub. No. U.S.-2003-0126641-A1.

Alternative methods for introducing DNA into plant cells include, for example, the use of liposomes, electroporation, chemicals that increase free DNA uptake, DNA delivery via microprojectile bombardment, and transformation using viruses or other biological agents.

Plant transformation vectors may be derived from the Ti or root-inducing (Ri) plasmids from Agrobacterium tumifaciens (FIG. 7). Fungal or viral vectors are disclosed, for example, by Herrera-Estrella et al. (1983), Bevan (1984), Klee et al. (1985) and EPO patent application Pub. No. 120,516.

Additional examples of Ti vectors suitable for transformation of dicotyledons include plasmid vectors PMON 200, pMON, pMON 505, pMON 526 and PMON 17227 (U.S. patent application Pub. No. US-2003-0126641-A1).

Additionally, vectors known in the art are capable of stably transforming the chloroplast genome (U.S. patent application Pub. No. US-2003-0041353-A1). Such vectors include chloroplast expression vectors such as pUC, pBlueScript, pGEM, and others identified in U.S. Pat. Nos. 5,693,507 and 5,932,479. A universal integration and expression vector that is competent for stably transforming the chloroplast genome of different plant species has been described in International Patent Application Pub. No. WO99/10513.

DNA constructs formed from gene fusions can be delivered to plant cells using either DNA viruses or RNA viruses as transport vehicles.

An example of a double-stranded DNA virus in plants is the cauliflower mosaic virus (CaMV). These viral genomes may be used as vectors for inserting foreign DNA into plant cells). For example, U.S. Pat. No. 4,407,956 describes the use of cauliflower mosaic virus DNA as a vector for introducing foreign DNA into a plant cell and its modification to extend the host range of the virus beyond the Cruciferae.

An example of a single-stranded DNA virus are the Gemini viruses. Gemini viruses are of interest to genetic engineers, since they can infect monocots where their DNA enters the nucleus. The bean golden mosaic-virus (BGMV), the cassava latent virus (CLV), the tomato golden mosaic virus (TGMV), the maize streak virus (MSV), and the abutilon mosaic virus belong all to the Gemini-virus family (http://www.biologie.uni-hamburg.de/b-online/e35/35c.htm).

An example of a recombinant RNA virus is provided in U.S. Pat. No. 5,804,439 which describes encapsidation of genetically engineered viral sequences in heterologous, preferably rod-shaped coat, protein capsids, which are expansible.

An example of an expansible rod-shaped virus is tobacco mosaic virus (TMV) which is suited for introducing exogenous genetic material into lettuce, spinach, tomato, potato, beans and tobacco (U.S. Pat. No. 6,503,732). Another example of an RNA virus is the Dianthovirus which includes but is not limited to red clover necrotic mosaic virus, carnation ringspot virus, sweet clover necrotic mosaic virus and furcraea necrotic streak virus (U.S. Pat. No. 6,433,248).

In embodiments of the invention, an RNA fusion may be made between a viroid RNA sequence and an RNA expressed by a gene fragment in the cell nucleus which can be prepared as a fusion RNA in vitro for incorporation into an RNA virus. The product RNA would then be suitable for splicing with an additional RNA fragment to create an intact mRNA for translation into the target protein.

Alternately, naked DNA can be introduced into plant cells by liposome delivery, mechanical (biolistic) methods such as use of a gene gun. Additional methods for transforming plant cells or animal cells include electroporation or transfection of DNA by direct contact with cells or isolated chloroplasts using salts such as CaCl₂ or lipid carriers such as lipofectin.

When adequate numbers of cells (or protoplasts) containing the gene fragments or RNA for expressing the target protein are obtained, the cells (or protoplasts) are regenerated into whole plants. Choice of methodology for the regeneration step is not critical, with established protocols being available for hosts from Leguminosae (alfalfa, soybean, clover, etc.), Umbelliferae (carrot, celery, parsnip), Cruciferae (cabbage, radish, canola/rapeseed, etc.), Cucurbitaceae (melons and cucumber), Gramineae (wheat, barley, rice, maize, etc.), Solanaceae (potato, tobacco, tomato, peppers), various floral crops, such as sunflower, and nut-bearing trees, such as almonds, cashews, walnuts, and pecans. See for example, Ammirato et al., Handbook of Plant Cell Culture—Crop Species, MacMillan PubI. Co. (1984); Fromm, Methods in Enzymology 153: 351-366(1990); Vasil et al., Bio/Technology 8: 429-434(1990); Vasil et al., Bio/Technology 10:667-674(1992); Hayashimoto, Plant Physiol. 93:857-863(1990); and Datta et al., Bio/Technology 8, 736-740(1990).

(6) Determining the Location of Split Gene Sequences in the Relevant Cellular Compartment

The location of the reconstituted split genes can be determined in a number of ways. For example, different antibiotic resistance markers can be used to detect whether gene fragments have been successfully introduced into the nucleus or cytoplasm (see Examples II and IV).

Once the gene fragments have been successfully introduced into a target cell, the presence of intact mRNA, which is the trans-spliced product, can be detected by the activity of the translated protein. For example, EPSPS protein functions in the chloroplast to provide glyphosate-resistance. Glyphosate resistance is indirect evidence for the proper location of the reconstituted EPSPS protein.

If a direct method is preferred, a marker such as the GFP or fluorescently-tagged antibodies that cross-react with the target protein can be used. The intracellular location of the fluorescent marker or the fluorescent antibody-bound target protein can be determined by confocal microscopy.

The examples and figures describe in detail how bacterial models can be used to test the split gene technology for feasibility in plants (Examples I-III). The bacterial model system is effective for demonstrating RNA trans-splicing and does not require measuring the activity of the protein in bacteria. Use of bacterial model systems obviate the need to initially test the splitting of a protein and subsequent reconstitution in more time consuming experiments in plants. Once a useful split coding sequence has been developed and reconstitution shown in bacteria, the experiments can be repeated in plants as described for intact soluble modified GFP from two fragments and for EPSPS. Once reconstituted, smGFP acts as a marker protein that permits the visual determination of the localization of the intact protein product.

Publications, patents and patent applications cited herein are incorporated by reference to the same extent as if each individual publication or patent was specifically and individually indicated to be incorporated by reference.

### EXAMPLES

### Example I

### Use of a Trans-Splicing Ribozyme to Join the RNA Encoding Two Protein Fragments in Bacterial Cells

A trans-splicing ribozyme (Tetrahymena Intron 1 ribozyme) is used to join the RNA encoding two GFP protein fragments in E. coli cells. The spliced fragments express the active protein smGFP which is detected both by fluorescence and by Western Blot analysis (see FIGS. 2 and 3).

The trans-splicing ribozyme DNA based on the T. thermophila group I self-splicing intron is obtained using overlapping oligonucleotides followed by a PCR. The overlapping oligonucleotides used are:

| | |
|---|---|
| gccatggaactcgagcccgctcttccaaaagttatcaggcatgcacctggta; | (SEQ ID NO:7) |
| gattgcatcggtttaaaaggcaagaccgtcaaattgcgggaaaggggtcaaca; | (SEQ ID NO:8) |
| tcaggggaaactttgagatggccttgcaaagggtatggtaataagctgacgg; | (SEQ ID NO:9) |
| gccaagtcctaagtcaacagatcttctgttgatatggatgcagttcacagact; | (SEQ ID NO:10) |
| atgtattcttctcataagatatagtcggacctctccttaatgggagctagcgg; | (SEQ ID NO:11) |
| gagccgctgggaactaatttgtatgcgaaagtatattgattagttttggagt; | (SEQ ID NO:12) |
| gctgcagaggcggccgccaaaggaccgaatgcgagtactccaaaactaatcaa; | (SEQ ID NO:13) |
| ttagttcccagcggctccagtgttgcatcacttcatccgctagctcccattaa; | (SEQ ID NO:14) |
| ttatgagaagaatacatcttccccgaccgacatttagtctgtgaactgcatc; | (SEQ ID NO:15) |
| ttgacttaggacttggctgcgtggttaggaccatgtccgtcagcttattacca; | (SEQ ID NO:16) |
| ctcaaagtttcccct gagacttggtactgaacggctgttgacccctttcccg; | (SEQ ID NO:17) |
| and | |
| tttaaaccgatgcaatctattggtttaaagactagctaccaggtgcatgcctg. | (SEQ ID NO:18) |

The oligonucleotides are annealed in a solution containing 1 μM of each oligonucleotide and 1XEcoPol buffer (10 mM Tris-HCl, pH 7.5, 5 mM MgCl2, 7.5 mM dithiothreitol). The annealing solution is heated to 80° C. for 5 min. followed by 10 min. on ice. The annealed primers are treated with the Klenow fragment (New England Biolabs, Inc., Beverly, Mass.) for 15 min. at room temperature after which the annealed and extended oligonucleotides are purified using a QIAquick PCR purification kit (Qiagen). 8 μL of the annealed oligonucleotide solution are used in a 100 μL PCR reaction using SEQ ID NO:7 and SEQ ID NO:13 as primers. The ribozyme amplicon is ligated into the pCR-bluntII TOPO vector (Invitrogen). E. coli (strain DH5-a) chemically competent cells (Invitrogen, catalog number 12297016) are transformed by heat shock. The transformed E. coli cells are plated onto LB agar plates containing 50 μg/mL kanamycin. After overnight incubation at 37° C. colonies are picked and grown in individual 5 mL cultures overnight at 37° C. DNA from each culture are purified using a DNA purification mini-prep kit (Qiagen). Correct constructs are determined by DNA sequencing (SeqWright, Inc., Houston, Tex.). The new plasmid are termed pRib-TOPO1. The NcoI to PstI fragment from pRib-TOPO1 corresponding to the Rib gene are subcloned into the same sites in pTXB3 (New England Biolabs, Inc., Beverly, Mass.) and the plasmid is termed pRib1. A cassette corresponding to the necessary antisense region of the Ribozyme (FIG. 3) is inserted into the NcoI to SapI sites of pRib1. The cassette is made by annealing two oligonucleotides: CATGCACCAGGATTTGTCGTGAGGCCTGAGTTCAGACCGGTGAATT GAGAACACGGTAAGA (SEQ ID NO:19); and TTTTCTTACCGTGTTCTCAATTCACCGGTCTGAACTCAGGCCTCACGA CAAATCCTGGTG (SEQ ID NO:20).

The new plasmid is termed pRib2.

The 5′ end of the smGFP gene is amplified from plasmid psmGFP (Arabidopsis Biological Resource Center, stock number CD3-326) using primers: GGCCCATGGGTAAAGGAGAAGAACTTTTCA (SEQ ID NO:21) and GGCACCGGTTTTCTACGATMGAGAAAGTAGTGACAAG (SEQ ID NO:22). The smGFP amplicon is cloned into a pCR-bluntII-TOPO vector and subjected to DNA sequencing. The new plasmid is termed pGFP-TOPO10. In addition, the 3′ end of the smGFP gene is amplified using oligonucleotides GGCGAATGCGGGTGTTCAATGCTTTTCAAG (SEQ ID NO:23) and GAAGCGGCCGCTTATTTGTATAGTTCATCCATG (SEQ ID NO:24) and psmGFP as a template. The amplicon is cloned into a pCR-bluntII-TOPO vector and sequenced. The new plasmid is termed pGFP-TOPO11.

The DNA equivalent of the trans-splicing ribozyme is fused to the 5′ end of the gene fragment encoding the C-terminal fragment of soluble modified GFP (smGFP) (see Davis, Plant Mol. Biol., 36(4):521-528 (1998)). This is accomplished by subcloning the BsmI to NotI fragment containing the partial smGFP gene from pGFP-TOPO11 into the same sites in pRib2. The new plasmid is termed pRib3.

The NcoI to PstI DNA fragment containing the ribozyme is subcloned from pRib3 into the same sites in plasmid pKEB12 (Chen, L., et al, Gene (2001) 263:39). The new construct is termed pRib4. A second construct is made by subcloning the NcoI to AgeI fragment containing the partial smGFP gene from pGFP-TOPO10 into the same sites in pTXB3 (New England Biolabs, Inc., Beverly, Mass.). The new plasmid is termed pTGFP1. The plasmids contained the compatible p15a, pRib4, and colE1, pTGFβ1, origins of replication.

The pTGFP1 plasmid uses the IPTG inducible T7 promoter to control transcription (Studier, F. W., et al, Methods Enzymol. 185:60-89 (1990) and Dubendorff, et al. J. Mol. Biol. 219:45-59 (1991)) while plasmid pRib4 uses the IPTG inducible Ptac promoter (Chen, L., et al, Gene (2001) 263:39). Each plasmid contain different antibiotic resistance markers so that they can both be maintained in the same E. coli cell.

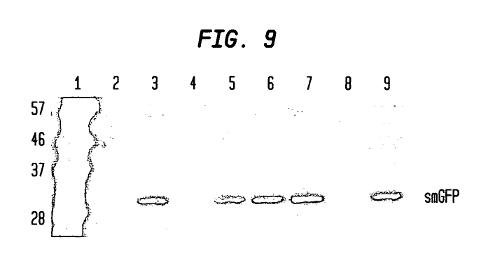

Following transformation of competent E. coli cells with both pRib 4 and pTGFP1 plasmids the cells are monitored for splicing activity by observing them under a microscope set up to detect smGFP fluorescence (for protocols on GFP use, see Sullivan, Kevin F. and Kay, Steve A., Methods in Cell Biology, V. 58: Green Fluorescent Proteins). In addition, Western Blot analysis using anti-smGFP antibody is used to confirm the presence of the smGFP protein based upon the observed migration on SDS-PAGE (FIG. 9).

Routine protein science techniques described herein are described in Current Protocols in Protein Science, Series Editor Virginia Benson Chanda, John Wiley & Sons, Inc.). Additional references include, Sambrook, J. and Russell, David W., Molecular Cloning, 3rd Edition, Cold Spring Harbor Laboratory Press, Cold Spring Harbor, N.Y. (2001).

### EXAMPLE II

### Expression of smGFP from a Viroid ASBVd DNA-GFP Fusion

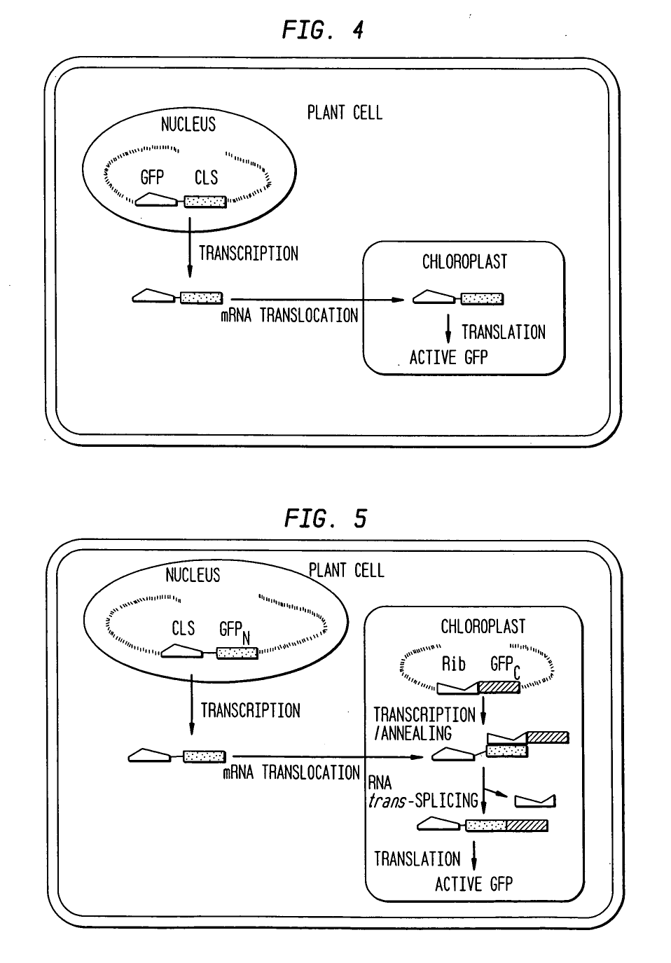

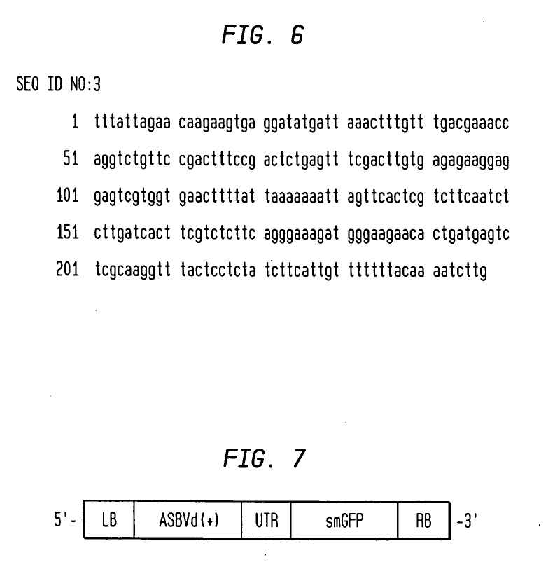

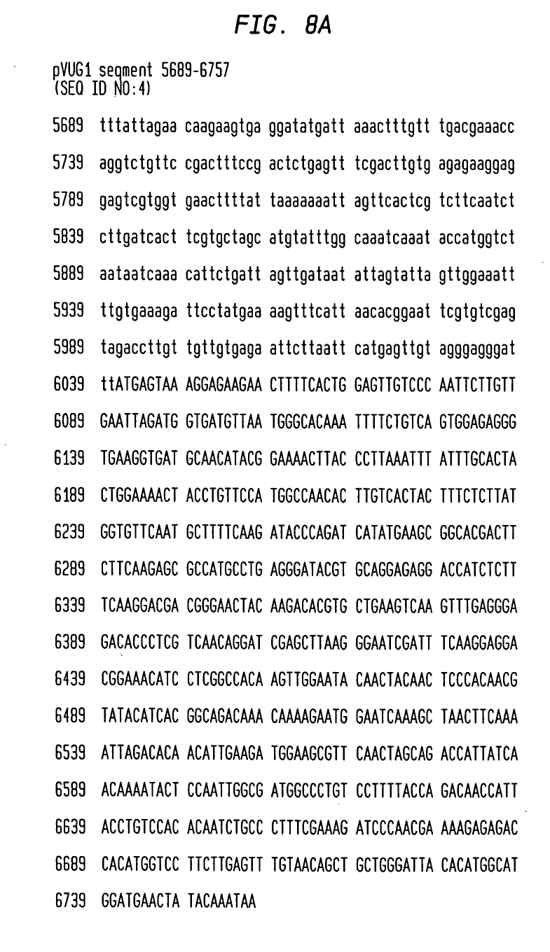

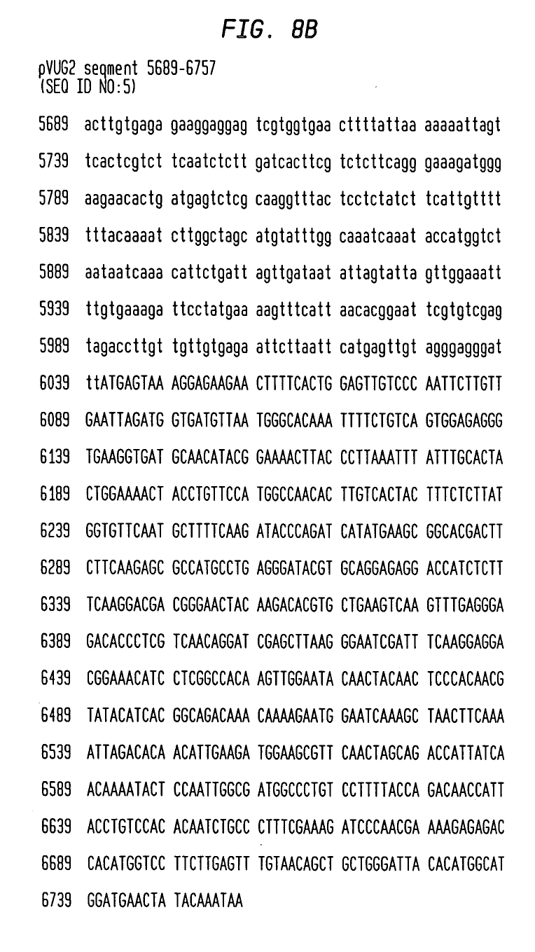

The expression of DNA corresponding to viroid ASBVd fused to the gene for smGFP (see FIGS. 4, 7, and 8) in plant cells was tested as a prerequisite of expression of a splice product in the chloroplast.

Using the method illustrated in FIGS. 4 and 10 and the GFP constructs described in Example I, the ability of a viroid sequence to permit RNA localization to the chloroplast was investigated in tobacco plant cells. The DNA encoding the smGFP was fused to DNA corresponding to a CLS, in this case a partial ASBVd, in an E. coli cloning vector, E. coli plasmid pTWIN1 (available from New England Biolabs, Inc., Beverly, Mass.), using standard molecular biology procedures (Sambrook, J. & Russell, D. W., Molecular Cloning: A Laboratory Manual, 3^{rd} Edition, Cold Spring Harbor Laboratory Press: Cold Spring Harbor, N.Y. (2001).

The DNA equivalent of a partial ASBVd sequence (nucleotides 84-247) was prepared as follows. The following primers were annealed in solution:

| | |
|---|---|
| tttattaaaaaaattagttcactcgtcttcaatctcttgatcacttcgt; | (SEQ ID NO:25) |
| ctaatttttttaataaaagttcaccacgactcctccttctctcacaagt; | (SEQ ID NO:26) |
| gtctagaacttgtgagagaaggaggagtcgtggtgaact; | (SEQ ID NO:27) |
| ggaagaacactgatgagtctcgcaaggtttactcctctatcttcattgtt; | (SEQ ID NO:28) |
| ggctagccaagattttgtaaaaaaacaatgaagatagagg; | (SEQ ID NO:29) |
| and | |
| ctcatcagtgttcttcccatctttccctgaagagacgaagtgatcaagaga. | (SEQ ID NO:30) |

The annealing solution contained 1 μM of each oligo and 1XEcoPol buffer (10 mM Tris-HCl, pH 7.5, 5 mM MgCl2, 7.5 mM dithiothreitol). The annealing solution was heated to 80° C. for 5 min. followed by 10 min. on ice. The annealed primers were treated with the klenow fragment (New England Biolabs, Inc., Beverly, Mass.) as per the manufactures recommendations for 15 min. at room temperature after which the annealed and extended oligonucleotides were cleaned up using a QIAquick PCR purification kit as per the manufacturers instructions (Qiagen). This annealed sample was used as a template in a DNA amplification reaction (polymerase chain reaction) using SEQ ID NO:27 and SEQ ID NO:29 as primers. The amplified partial ASBVd DNA was cloned into a pCR-bluntII TOPO vector (Invitrogen). This plasmid was termed pVir-TOPO2. The DNA for the Rubisco LSU 5′ UTR was generated by annealing the following oligonucleotides:

| | |
|---|---|
| gggctagcatgtatttggcaaatcaaataccatggtctaa; | (SEQ ID NO:31) |
| agttgataatattagtattagttggaaattttgtgaaagattcctatgaaaa; | (SEQ ID NO:32) |
| tcgtgtcgagtagaccttgttgttgtgagaattcttaattcatgagttgtag; | (SEQ ID NO:33) |
| ccgctcttcacataaatccctccctacaactcatgaatta; | (SEQ ID NO:34) |
| aggtctactcgacacgaattccgtgttaatgaaacttttcataggaatcttt; | (SEQ ID NO:35) |
| and | |
| tactaatattatcaactaatcagaatgtttgattattagaccatggtatttg. | (SEQ ID NO:36) |

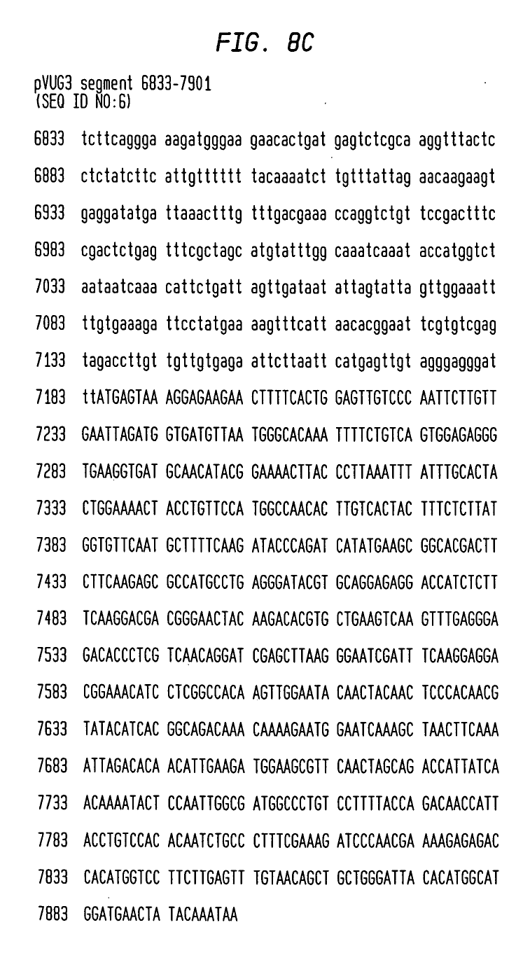

The annealing solution contained 1 μM of each oligo and 1XEcoPol buffer (10 mM Tris-HCl, pH 7.5, 5 mM MgCl2, 7.5 mM dithiothreitol). The annealing solution was heated to 80° C. for 5 min. followed by 10 min. on ice. The annealed primers were treated with the klenow fragment (New England Biolabs, Inc., Beverly, Mass.) for 15 min. at room temperature as per the manufacturers recommendations after which the annealed and extended oligos were cleaned up using a QIAquick PCR purification kit as per the manufacturers instructions (Qiagen). This sample was used as a template in a DNA amplification reaction using SEQ ID NO:31 and SEQ ID NO:34 as primers. The amplified UTR DNA was cloned into a pCR-bluntII TOPO vector (Invitrogen). This plasmid was termed pUTR-TOPO1. The soluble modified green fluorescent protein was amplified from plasmid psmGFP (Arabidopsis Biological Resource Center, stock number CD3-326) using primers: GGGCTAGCGCTGCTCTTCCATGAGTAAAGGAGMGMCAACTTT (SEQ ID NO:37) and GGCTGCAGGAGAGCTCTTATTTGTATAGTTC (SEQ ID NO:38). The amplicon was cloned into a pCR-bluntII TOPO vector (Invitrogen). The plasmid was termed pGFP-TOPO1. Plasmid pUV2 was created by subcloning the NheI to AlwNI fragment from pVir-TOPO2 into the same sites in pUTR-TOPO1. Plasmid pGVD2 was created by subcloning the NheI to PstI fragment from pGFP-TOPO1 into the same sites in pTWIN1 (New England Biolabs, Inc., Beverly, Mass.). This plasmid was termed pGVD2. Plasmid pVUG2 (FIG. 8B) was created by subcloning the XbaI-SapI fragment from pUV2 into the same sites in pGVD2.

N. tabacum leaf sections were treated with the transformed A. tumefaciens then placed on a cocultivation medium (1×MS salts, 3% sucrose, 2 mg/L alpha-naphthaleneactic acid, 0.5 mg/L 6-benzylaminopurine). These were incubated for 2-3 days in the dark at 28° C. Leaf sections were selected using a medium composed of 1×MS salts, 3% sucrose, 2 mg/L alpha-naphthaleneactic acid, 0.5 mg/L 6-benzylaminopurine, 500 mg/L carbeniciilin, and 100 mg/L kanamycin (Chin, G., et al., Proc. Natl. Acad. Sci. USA 100:4510(2003)).

Leaf samples were analyzed for expression of GFP as follows. Leaf cell extracts were prepared by homogenization as described on page 4511 of Chin, et al (2003) PNAS, 100:4510. Samples of the homogenized plant cells were analyzed on a Western Blot using a monoclonal GFP antibody (Hoffman-LaRoche, White Township, N.J.). Western Blots were performed as described in Sambrook, J. & Russell, D. W., Molecular Cloning: A Laboratory Manual, 3^{rd} Edition, Cold Spring Harbor Laboratory Press: Cold Spring Harbor Laboratory, NY (2001). The results are provided in FIG. 9 showing that five out of seven leaf samples expressed GFP.

Alternatives to the above analysis include, (a) obtaining chloroplasts from the homogenized leaf extracts and purifying the chloroplasts on a Percoll gradient (Maliga, P., et al.,” Cold Spring Harbor Laboratories, Cold Spring Harbor, N.Y. (1995)). The chloroplasts can then be broken open by boiling with SDS sample buffer and samples loaded onto a gel for analysis by Western Blot or (b) localizing smGFP fluorescence to the chloroplasts using a confocal microscope (Methods in Cell Biology, Volume 58: Green Fluorescent Proteins; edited by Kevin F. Sullivan and Steve A. Kay).

### EXAMPLE IIIa

### Expression of EPSPS in Tobacco After RNA Trans-Splicing

The feasibility of RNA trans-splicing and translocation to produce an herbicide resistant N. tabacum from compartmentalized gene fragments is described here using a mutant form of 5-enolpyruvylshikimate-3-phosphate synthase (EPSPS), (Chen, et al., Gene, 263(1-2):39-48(2001) and Stalker, D. M., et al., J. Biol. Chem. 260:4724(1985)) to generate tobacco plants with resistance to the herbicide glyphosate (See FIG. I). The methodological approach is consistent with Examples I, II, IV and V.

A gene fusion is formed for insertion into the nucleus of the plant cell. The nuclear gene fusion contains a CLS, a UTR, and the 5′ end of the EPSPS gene. The 5′ end of the EPSPS gene is amplified by PCR using primers: GGGCTAGCGCTGCTCTTCCATGGAATCCCTGACGTTACAA (SEQ ID NO:39); and GGCCTGCAGGAGCTCTTTCTGCCACCTGGAGAGTGATACTGTT (SEQ ID NO:40) from plasmid pPMG1 (available from the American Type Culture Collection, ATCC number 39256). The amplicon is cloned into pCR-bluntII TOPO and the sequence verified (SeqWright, Inc., Houston, Tex.). This plasmid is termed pEPS200. The NheI to PstI fragment containing the partial EPSPS gene from pEPS200 is subcloned into the same sites in pTWIN1. The new plasmid is termed pEPS201. The pUV2 XbaI to SapI fragment containing the UTR sequence is subcloned into the same sites in pEPS201 creating plasmid pEPS202. To get the nuclear transformation vector the XbaI to SacI fragment from pEPS202 containing the CLS sequence is subcloned into the same sites in pBI121 to create pEPS203. The CLS-UTR-EPSPS fusion is integrated into the nuclear genome of N. tabacum as described in Example II.

A trans-splicing ribozyme construct specific for EPSPS is created by inserting a cassette composed of oligonucleotides CATGTTGCCAAATGTTTGMCGATCGGGGAAATTCGAGCTCGAATTG TGATAGCCGCCTGG; and TTTCCAGGCGGCTATCACAATTC GAGCTCGAATTTCCCCGATCGTTCAMCATTTGGCAA (SEQ ID NO:41) and inserting this cassette into the NcoI to SapI sites in pRib1. This plasmid is identified as pRib5. The 3′ end of the EPSPS gene is amplified by PCR using primers GGCGAATGCGCGCTATCTGGTCGAGGG (SEQ ID NO:42); and GAAGCGGCCGCACCGGTTTAGGCAGGCGTACTCATT (SEQ ID NO:43) and pPMG1 as a template.

The amplicon is cloned into a pCR-bluntII-TOPO vector and sequenced. The new plasmid is called pEPSPS204. Plasmid pEPSPS205 is created by subcloning the BsmI to NotI fragment containing the EPSPS gene fragment from pEPSPS204 into the same sites in pRib5. The chloroplast transformation vector pEPSPS206 is created by subcloning the PmeI to AgeI fragment containing the ribozyme from pEPSPS205 into the same sites in plasmid p226alg (Chin, G., et al., Proc. Natl. Acad. Sci. USA 100:4510(2003)).

Chloroplasts are transformed using a biolistic PDS-1000 helium particle delivery system (Bio-Rad) as described previously (Chin, G., et al, (2003) Proc. Natl. Acad. Sci. USA 100:4510). 30 mg of gold particles (0.7 μM) is precipitated. The particles are re-suspended with 1 mL of 70% ethanol and 1 mL of water. The gold particles are pelleted by centrifugation and the supernatant is removed. The particles are re-suspended with 5 μL of pEPSPS206 (1 μg/μL). To this was added 50 μL CaCl₂ (2.5 M) and 20 μL spermidine (0.1 M, free base, tissue grade). The solution is mixed and the particles pelleted by centrifugation (2-3 s) in a microcentrifuge. The supernatant is removed and the pellet is washed with 70% ethanol and re-suspended with 48 μL of ethanol (100%).

Healthy leaves from 4-5 week old plants (N. tabacum already containing the CLS-UTR-EPSPS gene fusion in the nuclear genome as described above) are harvested and placed onto TSMCK media (1×Murashige and Skoog (MS) basal salt, 1XB5 vitamin mix, 0.005 mg/L kinetin, and 4 mg/mL p-chlorophenoxyacetic acid). These are incubated overnight at 28° C. in the dark. The pEPSPS206 suspension (8 pL) is bombarded at 1300 psi of helium onto the leaf containing plates. The bombarded leaves are left for 2 days in the light at 28° C. after which they are cut and moved to MST5 media (premixed MS media with sucrose and agar (Sigma), 0.1 mg/L a-naphthaleneacetic acid, and 1 mg/L 6-benzylaminopurine) supplemented with 500 mg/L spectinomycin. The leaf sections are transferred to new MST5 media after 2 weeks. Small green spots appear after 2-3 weeks from which multiple shoots emerge. The shoots (10-15 mm in length) are transferred to MS media for rooting (in Magenta boxes). The presence of the full-length EPSPS protein in the transgenic N. tabacum is determined by Western Blot analysis using anti-EPSPS antibody. Furthermore, The 4 week old transgenic plants are subjected to glyphosate treatment to determine whether the EPSPS protein is functional.

### EXAMPLE IIIb

### Expression of ALS in Tobacco After RNA Trans-Splicing

The feasibility of RNA trans-splicing and translocation to produce an Sulfonylurea herbicide resistant N. tabacum from compartmentalized gene fragments is shown here. The methodological approach is consistent with Examples I, II, IIIa, IV and V.

A mutant form of ALS, (Sun, L., et al., App. Environ. Microbiol. 67:1025(2001); Bernasconi, P., et al., J. Biol. Chem. 270:17381(1995); LaRossa, R. A., & Smulski, D. R., J. Bacteriol. 160:391(1984); and Yadav, N., et al., Proc. Natl. Acad. Sci. USA 83:4418(1986)) was used to generate tobacco plants with resistance to the sulfonylurea herbicides.

A pool of cDNA from the commercially available Pioneer 3180 IR corn is created as described previously (Sun, L., et al, App. Environ. Microbiol. 67:1025(2001)). The site to split the protein is amino acid 398 of the mutant form of corn acetolactate synthase (cALS(Ala56Thr), Greaves, J. A., et al, (1993) Proceedings of the 48^{th} Annual Corn and Sorghum Industry Research Conference, pp 104-118) present in Pioneer 3180 IR corn. This is close to the site used previously (amino acid residue 397) as described elsewhere (Sun, L., et al, (2001) App. Environ. Microbiol. 67:1025). The shift to amino acid residue serine 398 is necessary to provide a T (a U in the mRNA) at the site of RNA trans-splicing. The gene fragment corresponding to the 5′ end of cALS(Ala56Thr) is amplified by PCR using the following primers: GGGCTAGCGCTGCTCTTCCATGGCCACCGCCGCCG (SEQ ID NO:44) and GGCCTGCAGGAGCTCTTTCTTTCATGTGCTTCCTTCAAGMGA (SEQ ID NO:45). The template is the cDNA pool from Pioneer 3180 IR corn prepared as described previously (Sun, L., et al., App. Environ. Microbiol. 67:1025(2001)). The PCR product is cloned into pCR-bluntII TOPO. The plasmid is termed pcALS1. The plasmid is subjected to DNA sequencing to verify that the cALS(Ala56Thr) gene fragment is present and not the wild type cALS gene fragment.

The NheI to PstI fragment containing the partial cALS(Ala56Thr) gene from pcALS1 is subcloned into the same sites in pTWIN1. The new plasmid is termed pcALS2. The pUV2 XbaI to SapI fragment containing the UTR sequence is subcloned into the same sites in pcALS2 creating plasmid pcALS3. To get the nuclear transformation vector the XbaI to SacI fragment from pcALS3 containing the CLS sequence is subcloned into the same sites in pBI121 to create pcALS4. The CLS-UTR-EPSPS fusion is integrated into the nuclear genome of N. tabacum as described in Example II.

A trans-splicing ribozyme construct specific for cALS(Ala56Thr) is created by inserting a cassette composed of oligonucleotides

| | |
|---|---|
| CATGTTGCCAAATGTTTGAACGATCGGGGAAATTCGAGCTCGAATTGTTTCTTTCGTGTGC; | (SEQ ID NO:46) |
| and | |
| TTTGCACACGAAAGAAACAATTGGAGCTCGAATTTCCCCGATGGTTCAAACATTTGGCAA. | (SEQ ID NO:47) |

The cassette is inserted into the NcoI to SapI sites in pRib1. This plasmid is termed pcALS5. The 3′ end of the cALS(Ala56Thr) gene is amplified by PCR using primers GGCGAATGCGCAAAGAAGAGCTTTGACTTTG (SEQ ID NO:48) and GAAGCGGCCGCACCGGTTCAGTACACAGTCCTGCC (SEQ ID NO:49) and the cDNA pool from Pioneer 3180 IR corn as a template. The amplicon is cloned into a pCR-bluntII-TOPO vector and subjected to DNA sequencing. The new plasmid is termed pcALS6. The complete cALS(Ala56Thr) and Rib vector, pcALS7, is created by subcloning the BsmI to NotI fragment containing the cALS(Ala56Thr) gene fragment from pcALS6 into the same sites in plasmid pcALS5. The chloroplast transformation vector, pcALS8, is created by subcloning the PmeI to AgeI fragment from pcALS7 into the same sites in plasmid p226alg (Chin, G., et al., Proc. Natl. Acad. Sci. USA 100:4510(2003)).

Chloroplasts are transformed using a biolistic PDS-1000 helium particle delivery system (Bio-Rad) as described previously (Chin, G., et al, (2003) Proc. Natl. Acad. Sci. USA 100:4510). 30 mg of gold particles (0.7 μM) are precipitated. The particles are mixed with 1 mL of 70% ethanol and 1 mL of water. The gold particles are pelleted by centrifugation and the supernatant is removed. The particles are re-suspended with 5 μL of pcALS8 (1 μg/μL). To this is added 50 μL CaCl₂ (2.5 M) and 20 μL spermidine (0.1 M, free base, tissue grade). The solution is mixed and the particles pellet by centrifugation (2-3 s) in a microcentrifuge. The supernatant is removed and the pellet is washed with 70% ethanol and re-suspended with 48 μL of ethanol (100%).

Healthy leaves from 4-5 week old plants (N. tabacum already containing the CLS-UTR-cALS(Ala56Thr) gene fusion in the nuclear genome as described above) are harvested and placed onto TSMCK media (1×Murashige and Skoog (MS) basal salt, 1XB5 vitamin mix, 0.005 mg/L kinetin, and 4 mg/mL p-chlorophenoxyacetic acid). These are incubated overnight at 28° C. in the dark. The pcALS8 suspension (8 μL) is bombarded at 1300 psi of helium onto the leaf containing plates. The bombarded leaves are left for 2 days in the light at 28° C. after which they are cut and moved to MST5 media (premixed MS media with sucrose and agar (Sigma), 0.1 mg/L a-naphthaleneacetic acid, and 1 mg/L 6-benzylaminopurine) supplemented with 500 mg/L spectinomycin.

The leaf sections are transferred to new MST5 media after 2 weeks. Small green spots appear after 2-3 weeks from which multiple shoots emerge. The shoots (10-15 mm in length) are transferred to MS media for rooting (in Magenta boxes).

The presence of the full-length cALS(Ala56Thr) protein in the transgenic N. tabacum is determined by Western Blot analysis using anti- cALS(Ala56Thr) antibody. Furthermore, the 4 week old transgenic plants are subjected to sulfometuron methyl (Supelco, Bellefonte, Pa.) treatment to determine whether the cALS(Ala56Thr) protein is functional.

### Example IV

### Investigation of Modifications of Viroid Sequences Which Improve RNA Localization to the Chloroplasts

The minimal viroid sequence necessary to permit RNA localization to the chloroplast was investigated using the methods described in Examples I,II, IIIa, and IIIb and FIGS. 5 and 10.

The split smGFP system described in Examples I and II is used to determine the suitability of various viroids and mutant viroids to act as chloroplast localization sequences. The DNA encoding the smGFP N-terminal fragment (smGFPN) is fused to DNA corresponding to a chloroplast localization sequence (CLS) in an E. coli cloning vector, E. coli plasmid pTWIN1 (New England Biolabs, Inc. Beverly, Mass.), using standard molecular biology procedures (Sambrook, J. & Russell, D. W., Molecular Cloning: A Laboratory Manual, 3^{rd} Edition, Cold Spring Harbor Laboratory Press: Cold Spring Harbor, N.Y. (2001). The 5′ UTR from the N. tabacum plastid encoded rubisco large subunit is fused between the CLS and the target gene (smGFP_{N}). This sequence directs translation in the chloroplast. This DNA fusion fragment is placed between the recombination sequences, present for example in a plasmid like pPZP100 (Maliga, P., et al., Methods in Plant Molecular Biology: A Laboratory Course Manual, Cold Spring Harbor Laboratories Press, Cold Spring Harbor, N.Y. (1995) or pBI121 (Chin, G., et al., Proc. Natl. Acad. Sci. USA 100:4510 (2003)) required for A. tumefaciens mediated nuclear transformation. This is also described in Example II.

A. tumefaciens is used to transform the nucleus of an N. tabacum plant with the DNA encoding the CLS-UTR-smGFPN fusion using an antibiotic to select for transformed plants (Zupan, Plant J. 23(1):11-28 (2000); Maliga, P., et al.; Methods in Plant Molecular Biology: A Laboratory Course Manual, Cold Spring Harbor Laboratories Press, (1995); Chin, G., et al, (2003) Proc. Natl. Acad. Sci. USA 100:4510) as in Example II.

In addition, the DNA encoding the smGFP C-terminal fragment (smGFP_{C}) is fused to the DNA equivalent of a trans-splicing ribozyme derived from the T. thermophila group I intron (Kohler, U., et al, J. Mol. Biol., 285(5):1935-1950 (1999)) and incorporated into the chloroplast genome of the N. tabacum plant containing the DNA for the CLS-UTR-smGFP_{N} fusion in the nucleus. The chloroplast transformation is performed using standard biolistic procedures (Klein, et al, Curr. Opin. Biotechnol., 4(5):583-590 (1993); Chin, G., et al., Proc. Natl. Acad. Sci. USA 100:4510 (2003)) and antibiotic selection (Chin, G., et al., Proc. Natl. Acad. Sci. USA 100:4510 (2003)) as in Examples IIIa and IIIb. Transcripts of the desired gene fusions are driven by promoters such as the PpsbA promoter in the chloroplast or by the cauliflower mosaic virus 35S promoter in the nucleus (Zupan, Plant J. 23(1):11-28 (2000); Maliga, P., et al., Methods in Plant Molecular Biology: A Laboratory Course Manual, Cold Spring Harbor Laboratories Press, Cold Spring Harbor, N.Y. (1995); Chin, G., et al., Proc. Natl. Acad. Sci. USA 100:4510 (2003); Fujisawa, Y., et al., Proc. Natl. Acad. Sci. USA 96:7575 (1999)).

The ability of the CLS to move mRNA from the cytoplasm into the plastid can be determined by looking for full length smGFP. This is accomplished by Western blot analysis of plant extract using an anti-smGFP antibody (Sambrook, J. & Russell, D. W., Molecular Cloning: A Laboratory Manual, 3^{rd} Edition, Cold Spring Harbor Laboratory Press: Cold Spring Harbor, N.Y. (2001). Also, properly folded, full-length smGFP is detected by fluorescence microscopy (Sullivan, Kevin F. and Kay, Steve A., Methods in Cell Biology, Volume 58: Green Fluorescent Proteins).

To determine optimal sequences for chloroplast localization, various viroid sequences are tested, including all the ASBVd-type (Group A) viroids. (http://subviral. med.uottawaca/cgi-bin/accueil.cgi?typeRNA=1). Furthermore, deletions and substitutions are made in the viroid sequences to determine the minimal sequence for each viroid that still operates as a CLS. Finally, fusions of different viroids or partial viroids are tested for the optimal ability for chloroplast localization. These viroid sequences can be incorporated into the experimental system by incorporating the new viroid sequence in place of the partial ASBVd sequence in pUV2 in the examples above (particularly Example II).

### Example V

### Application of Trans-Splicing In Maize for Expression of BT Toxin

Using the methods described in Examples I, II, IIIa, IIIb and IV and FIGS. 5 and 10, a split gene method is applied to maize. As in Example IV the target gene, in this case Bt toxin (Van Rie J, Int. J. Med. Microbiol. 290:463 (2000)), is divided into two fragments. Each fragment encodes a non-active, truncated form of Bt toxin, preferably derived from Cry1A(c) (Genbank Accession #U87397). A gene fusion is created in which ASBVd, the CLS in this example, is fused to the 5′ untranslated region from the N. tabacum plastid encoded rubisco large subunit (UTR) and to the truncated Bt toxin gene (Bt_{N}) encoding an inactive, N-terminal fragment of the toxin using standard molecular biology techniques (Sambrook, J. & Russell, D. W., Molecular Cloning: A Laboratory Manual, 3^{rd} Edition, Cold Spring Harbor Laboratory Press: Cold Spring Harbor Laboratory, NY (2001). The ASBVd-UTR-Bt_{N} gene fusion is introduced into the nucleus of maize (Moellenbeck, D. J., Nature Biotech. (2001) 19:668).

A second gene fusion consisting of a T. thermophila group I intron derived ribozyme (Rib) and a truncated Bt toxin gene (Bt_{C}) encoding an inactive, C-terminal Bt toxin protein fragment is generated. The Rib is designed to specifically trans-splice the Bt_{N} and Bt_{C} gene fragments together. The Rib-Bt_{C} gene fusion is placed into the chloroplast of the plant already containing the ASBVd-UTR-Bt_{N} gene fusion in its nucleus. The chloroplast transformation uses the technique eluded to by Dhingra, A. & Daniell, H. (Abstract #888 (2002) American Society of Plant Biologists, website http://abstracts.aspb.org/pb2002/public/P73/1191.html).

Upon transcription and translocation to the cytoplasm, the ASBVd-UTR-Bt_{N} mRNA will translocate to the chloroplast via the CLS (ASBVd in this case). In the chloroplast the ASBVd-UTR-Bt_{N} and Rib-Bt_{C} mRNA's will anneal through the Rib and Bt_{C} will be spliced, also via the Rib, onto the ASBVd-UTR-Bt_{N} transcript. At this point full-length, active Bt toxin is produced to protect the corn from pests.

What is claimed is:

1. A method for translocating an RNA into a chloroplast, the method comprising:
contacting the chloroplast with an RNA comprising a first RNA sequence and a second RNA sequence, the first RNA sequence consisting of a chloroplast localization sequence (CLS), the second RNA sequence characterized by its non-natural association with the first RNA sequence; and
translocating the RNA into the chloroplast.

2. A method according to claim 1, wherein the CLS shares substantial homology with a viroid.

3. A method according to claim 1, wherein the CLS consists of at least part of a viroid

4. A method according to claim 2 or 3, wherein the viroid is an Avsunvirodiae viroid.

5. A method according to claim 4 wherein the viroid is an Avocado sunblotch viroid.

6. A method according to claim 4, wherein the viroid is a peach latent mosaic virus.

7. A method according to claim 4, wherein the viroid is selected from chrysanthemum chlorotic mottle viroid and eggplant latent viroid.

8. A method according to claim 1, wherein the second RNA sequence encodes a whole or a part of a target protein.

9. A method according to claim 8, wherein the target protein is a herbicide-resistant protein.

10. A method according to claim 9, wherein the herbicide-resistant protein is selected from 5-enolpyruvylshikimate-3-phosphate synthase and acetolactate synthase.

11. A method according to claim 8, wherein the target protein is an insecticidal toxin.

12. A method according to claim 11, wherein the insecticidal toxin is a Bacillus thurigensis toxin.

13. A method according to claim 8, wherein the protein is a marker protein.

14. A method according to claim 13, wherein the marker protein is green fluorescent protein.

15. A method according to claim 1, wherein the protein is a metabolic enzyme.

16. A method according to claim 15, wherein the metabolic enzyme is fructose 1,6-bisphosphate aldolase.

17. A method according to claim 1, wherein the second RNA sequence has a length of less than 10 kb.

18. A method according to claim 1, wherein the RNA is a product of transcription of a DNA.

19. A method according to claim 18, wherein the DNA is located in the nucleus of a plant cell containing the chloroplast.

20. A method according to claim 18, wherein the DNA is located in the cytoplasm of a plant cell containing the chloroplast.

21. A method according to claims 19 or 20, wherein the DNA is introduced into the plant cell by a viral vector.

22. A method according to claims 19 or 20, wherein the DNA is introduced into the plant cell by a physical or chemical means.

23. A method according to claim 1, wherein the RNA is a product of RNA replication.

24. A method according to claim 23, wherein the RNA is introduced into cytoplasm of a plant cell containing the chloroplast by an RNA virus.

25. A method according to claim 1, wherein the RNA further comprises an untranslated region sequence located between the first RNA sequence and the second RNA sequence.

26. A method according to claim 1, further comprising a third RNA sequence encoding part or whole of a second protein.

27. A method according to claim 1, wherein the second RNA sequence in the RNA encodes a first part of a protein and wherein the chloroplast contains a second RNA, the second RNA comprising a first RNA sequence and a second RNA sequence wherein the first RNA sequence is a ribozyme sequence and the second RNA sequence encodes a second part of the protein.

28. A method according to claim 27, wherein the first RNA and the second RNA are trans-spliced to form an RNA capable of being translated into the protein.

29. A method according to claim 28, wherein the ribozyme is a self-splicing group I ribozyme.

30. A method according to claim 29, wherein the ribozyme is a Tetrahymena thermophila intron I trans-splicing ribozyme.

31. A method according to claim 27, wherein the second RNA is encoded by a DNA containing a gene fragment fused to a DNA sequence encoding the ribozyme.

32. A method for expressing a whole or a part of a target protein in a chloroplast, the method comprising:
contacting the chloroplast with an RNA comprising a first RNA sequence and a second RNA sequence, the first RNA sequence consisting of a chloroplast localization sequence, the second RNA sequence encoding a whole or part of the target protein so that the first RNA chaperones the second RNA into the chloroplast; and
(a) expressing the whole or part of the target protein in the chloroplast.

33. An RNA comprising: a first RNA sequence which is substantially homologous to a segment of an avocado sunblotch viroid (ASBVd) and is characterized by a chloroplast localizing activity and a second RNA sequence which when translated, corresponds to part or all of a protein.

34. An RNA according to claim 33, wherein the segment corresponds to at least 100 nucleotides of the ASBVd.

35. An RNA comprising: a first RNA sequence which corresponds to a viroid and is characterized by a chloroplast localization sequence and a second RNA sequence which when translated, corresponds to part or all of a protein.

36. A bacterial cell containing at least one RNA characterized in claim 33 or 35.

37. A plant cell containing at least one RNA characterized in claim 33 or 35.

38. A virus containing an RNA, or a DNA encoding the RNA of claim 33 or 35.

39. A plasmid containing a DNA sequence for transcribing the RNA of claim 33 or 35.

40. An RNA according to claim 33 or 35 wherein the protein is selected from a herbicide-resistant protein, a pesticide-resistant protein, a marker protein and a metabolic enzyme.

41. A method of expressing a protein in a plant so that undesired gene flow in the environment is prevented, comprising:
(a) introducing into the nucleus of the plant, a first DNA wherein the first DNA comprises a first DNA sequence and a second DNA sequence such that the first DNA sequence is transcribed to form a first RNA sequence having a chloroplast localization sequence and the second DNA sequence is transcribed to form a second RNA sequence encoding a first part of a protein;
(b) introducing into the chloroplast of the first plant, a second DNA, wherein the second DNA comprises a third DNA sequence and a fourth DNA sequence such that the third DNA sequence is transcribed to form a ribozyme and the fourth DNA sequence is transcribed to form a fourth RNA sequence encoding a second part of the protein;
(c) permitting transcription of the first DNA and its translocation into the chloroplast for trans-splicing of the second RNA sequence to the fourth RNA sequence for translation into the protein; and
(d) inhibiting undesired gene flow in the environment.

42. A method according to claim 41, wherein the first fusion protein of step (a) comprises a fifth DNA sequence which is transcribed to form a fifth RNA sequence which after localization in the chloroplast is spliced to a sixth RNA to form a replicase protein.

43. A plant cell according to claim 41, further comprising a replicase translated from an exogenous nucleic acid contained in the plant cell.
